(19) United States
(12) Patent Application Publication
(10) Pub. No.: US 2026/0252913 A1
(43) Pub. Date: Aug. 27, 2026

(54) SEQUENTIAL AND DIFFERENTIAL PROCESSING OF STACKED KNOWLEDGE BASES AND SUBJECT DATA TO GENERATE INTELLIGENT AND CUSTOM OUTPUTS

(71) Applicant: Color Health, Inc., Burlingame, CA (US)

(72) Inventors: Munir Al-Dajani, New York, NY (US); Kiril Hristov Kafadarov, Seattle, WA (US); Othman Laraki, Atherton, CA (US); Wendy McKennon, San Francisco, CA (US); Keegan Duchicela, San Diego, CA (US); Deanna Brockman, Luxembourg (LU); Amina Lazrak, Los Angeles, CA (US)

(73) Assignee: Color Health, Inc., Burlingame, CA (US)

(21) Appl. No.: 19/396,074

(22) Filed: Nov. 20, 2025

Related U.S. Application Data
(60) Provisional application No. 63/763,746, filed on Feb. 26, 2025.

(51) Int. Cl. G06N 5/022; G06F 18/21; G16H 50/70

(52) U.S. Cl. CPC G06N 5/022; G06F 18/217; G16H 50/70

(57) ABSTRACT

The present disclosure relates to sequential and differential processing of stacked knowledge bases to retrieve a subset of knowledge bases and generate outputs personalized for a condition of a subject, based on an input dataset received via an interface accessible through a user endpoint, by leveraging a large language expert (LLE). The techniques, as disclosed herein, may dynamically generate task-specific queries corresponding to variables associated with each action represented in a knowledge base of the subset of knowledge bases. In response to execution of the task-specific queries on supplemental data associated with the subject, a result set comprising predicted intermediate results may be generated by leveraging a large language model (LLM). The disclosed techniques may further generate a workup trajectory through progressively refining the actions through application of associated first-order logics based on the generated result set and output the workup trajectory on the interface to guide subject’s pre-treatment workup.

### CROSS-REFERENCE TO RELATED APPLICATIONS

This application claims the priority to and the benefit of U.S. Provisional Application Number 63/763,746, filed on Feb. 26, 2025, entitled “Sequential and Differential Processing of Stacked Knowledge Bases and Subject Data to Generate Intelligent and Custom Outputs”, which is hereby incorporated by reference in its entirety for all purposes.

### BACKGROUND

Healthcare across diverse disciplines relies on clinical expertise, which may be essential for making accurate diagnoses and developing effective treatment plans. Achieving such clinical expertise often demands years of training beyond medical school, including residency, fellowship programs, and sub-specialization to build proficiency in specific conditions or diseases. However, with the rapid evolution of medical knowledge, driven by increasing complexity of treatments and diagnostic tools, maintaining the widespread and consistent dissemination of clinical expertise has become increasingly challenging. In particular, rendering all healthcare providers, especially those in smaller, non-academic settings, with timely access to the current advancements and specialized knowledge, remains a significant hurdle. Inadequate or delayed access to clinical expertise may result in slower diagnosis, incomplete evaluations, and suboptimal treatment decisions, potentially worsening patient outcomes.

These challenges may further be exacerbated when patient records may have to be accessed across fragmented systems, often in disparate formats. Physicians may already be burdened with the task of manually retrieving relevant data from various patient records, while also having to align that data with evidence-based guidelines that are frequently updated to incorporate new research and clinical insights. These procedures not only consume considerable amounts of time but also increase the risk of decision-making errors. However, many traditional systems have been developed to address these challenges, the persistent need to interpret unstructured, heterogeneous data (e.g., clinical notes, diagnostic reports) and convert it into standardized concepts that align with clinical workflows remains unresolved. Consequently, adherence to up-to-date, guideline-driven care continues to be a challenge in healthcare environments.

### SUMMARY

Some aspects of the present disclosure relate to techniques for processing a set of knowledge bases and subject-specific data, based on an input dataset, to generate intelligent and customized outputs personalized for a subject by leveraging a large language expert (LLE). The LLE features a hybrid architecture of an artificial intelligence (AI) model that may synergistically combine large language model (LLM) capabilities with deterministic, rule-based reasoning. The deterministic, rule-based reasoning may be configured by the LLE through integration of an LLM with rule-based logics encoded in the set of knowledge bases.

Each knowledge base of the set of knowledge bases may be developed to include, at a granular level, a guideline corresponding to one or more actions associated with a particular condition. Each action of the one or more actions may further be linked to one or more variables that may inform or influence the action and a rule-based logic, represented as a first-order logic, defining protocol under which the action may be recommended. Each knowledge base of the set of knowledge bases may be developed by leveraging a first AI technique implemented by the LLE.

A subset of knowledge bases from the set of knowledge bases may be accessed to generate personalized outputs for the subject based on an input dataset. The input dataset may be accessed via an interface, which may be accessible through a user endpoint associated with a user. The user may include, for example, a primary care physician such as a non-specialist unfamiliar with the subject’s case or a general practitioner. The input dataset, accessed via the interface, may comprise one or more of: a diagnosis, a potential diagnosis, a medical condition, or a corresponding code associated with a given condition of the subject. In some aspects, the input dataset may further include supplemental data associated with the subject.

Based on the input dataset, each knowledge base of the set of knowledge bases may be parsed to identify a plurality of knowledge bases. Parsing each knowledge base may be performed by evaluating one or more branching positions corresponding to the one or more variables. The one or more variables may further inform or influence each action of the one or more actions defined in the knowledge base. The one or more branching positions may be evaluated by leveraging a second AI technique that may dynamically generate one or more task-specific queries for the one or more variables and execute each of the one or more task-specific queries on the input dataset. For instance, a task-specific query may be generated for a branching position to determine whether the subject has been diagnosed with colon cancer, and the task-specific query may then be executed on the input dataset, which may include a confirmed or suspected diagnosis of colon cancer for the subject.

In some aspects, one or more task-specific queries may be accessed from each knowledge base of the set of knowledge bases, wherein each task-specific query may be associated with a variable of the one or more variables defined in that knowledge base. The accessed one or more task-specific queries from each knowledge base may have been generated during a development phase of that knowledge base by leveraging the first AI technique. Upon execution, the accessed one or more task-specific queries may be dynamically aligned with the input dataset based on the given condition of the subject, enabling context-aware evaluation of the relevant variables. For example, a task-specific query such as “Has the subject been diagnosed with colon cancer?” may have been accessed corresponding to a variable representing diagnosis. If the input dataset includes structured or unstructured data indicating a confirmed or suspected colon cancer diagnosis, the task-specific query may be dynamically aligned with that information, activating a corresponding branching position.

The corresponding branching position may be activated, in run-time, as the branching position pertains to the given condition of the subject, enabling further evaluation of one or more nodes, or one or more related branching positions. The one or more nodes may correspond to the one or more actions recommended based on the activated branching position. Further, based on identification of the one or more related branching positions, the evaluation of these branching positions may proceed through an iterative process, as described herein. The iterative process may continue until all relevant branching positions may be satisfied, resulting in a mapped trajectory corresponding to the activated branching position.

The mapped trajectory may form part of a plurality of mapped trajectories, that may correspond to the plurality of knowledge bases, derived from the input dataset. The plurality of mapped trajectories may be configured through a plurality of individual workflows, depending on the structure and dependencies defined in each knowledge base. In some aspects, the plurality of individual workflows may run simultaneously. In some other aspects, the plurality of individual workflows may be configured sequentially—for instance, when a mapped trajectory or associated guideline depends on an outcome of a previously mapped trajectory.

Therefore, each mapped trajectory of the plurality of mapped trajectories may result in one or more actions that may be configured based on the given condition of the subject. The plurality of knowledge bases, defined through the plurality of mapped trajectories, may further be dynamically stacked to access the subset of knowledge bases. The dynamic stacking may be implemented by hierarchically prioritizing one or more knowledge bases forming the subset of knowledge bases from the plurality of knowledge bases, based on predefined levels of guideline authority.

In some aspects, the plurality of knowledge bases may comprise open-source guidelines, institutional protocols, or department-level rules. The dynamic stacking may prioritize department-level rules over institutional protocols, and institutional protocols over open-source guidelines. For example, for a given condition such as breast cancer, if a department issues its own protocol differing slightly from the institution’s general guidelines or the open-source guidelines, the department-specific protocol may be selected as part of the subset of the knowledge bases used to guide the one or more actions.

Upon accessing the subset of knowledge bases, supplemental data may be accessed from one or more data sources. The one or more data sources may include structured electronic health record (EHR) fields, clinical text notes, scanned documents, PDFs, or other heterogeneous medical artifacts, potentially comprising unstructured or inconsistent information. In some aspects, the supplemental data may be processed to generate subject records in a structured format suitable for processing in conjunction with the subset of knowledge bases of the set of knowledge bases, which may be developed in a structured manner by leveraging the first AI technique.

Further, the one or more task-specific queries relevant to each mapped trajectory representing a guideline stored in a knowledge base may be incrementally evaluated by leveraging the second AI technique implemented by the LLE. The incremental evaluation may comprise executing the one or more task-specific queries of each mapped trajectory on the supplement data associated with the subject. In some aspects, the one or more task-specific queries may also be executed on the input dataset. Based on the incremental evaluation, a predicted intermediate result may be generated by the second AI technique for each task-specific query of the one or more task-specific queries. The predicted intermediate result may include: (a) a response, comprising one of: “yes,” “no,” or “unknown”, to each task-specific query; (b) a description justifying the response; and (c) one or more citations referencing the input dataset or the supplemental data associated with the subject.

To improve the efficiency and interpretability of this query evaluation process, the system may further utilize a technique referred to as question bundling. In this approach, individual queries that operate on a common clinical concept are grouped together under a shared concept. The question for the shared concept encompasses the underlying data needed to answer all questions in its bundle. For example, questions related to a patient’s age (“Is the patient younger than 55?”, “Is the patient 60 or older?”) can all be answered from the shared concept question “What is the patient’s age?”. This bundling allows the LLE to derive multiple answers from a single shared concept, improving consistency, reducing cognitive burden in the user interface, and aligning more closely with how clinicians reason about patient information.

Some aspects of the present disclosure relate to aggregating the predicted intermediate result of each task-specific query associated with each knowledge base of the plurality of knowledge bases to generate a result set. The result set may comprise the predicted intermediate results corresponding to the one or more variables, which may be relevant to the input dataset, or the supplemental data associated with the subject. The result set may then be output via the interface that may be accessible through the user endpoint, enabling the user to conduct a review.

Some aspects of the present disclosure further relate to assigning a confidence metric to each predicted intermediate result of the result set, prior to outputting the result set on the interface. The confidence metric may be determined based on the one or more factors such as accuracy, reliability, and relevance of the predicted intermediate result in the context of the supplemental data or the input dataset. In particular, the confidence metric may quantify a degree of certainty associated with the predicted intermediate result, as generated by the second AI technique (e.g., the LLM). In some aspects, the confidence metric may assist the user in prioritizing a specific predicted intermediate result during the review process wherein results with lower values of confidence metric may warrant closer scrutiny compared to the ones with higher values of the confidence metric.

During the review process, the user may override the responses associated with the one or more predicted intermediate results of the result set. The overridden responses may be received via the interface that may be accessible through the user endpoint. The second AI technique may then be leveraged to update the corresponding predicted intermediate results based on the overrides. Updating each predicted intermediate result of the one or more predicted intermediate results may include: a) generating a revised justification that may align with the overridden response; and b) identifying one or more citations from the supplemental data or the input dataset that may support the overridden response. In some aspects, a justification may be provided by the user via the interface along with the overridden response and may further be incorporated as a citation to the predicted intermediate result.

According to some aspects, if the user overrides a predicted intermediate result that may have been assigned a high value of the confidence metric based on the supplemental data associated with the subject, an additional processing may be triggered based on the override. Specifically, the overridden predicted intermediate result may be output via an interface accessible through an expert endpoint for a secondary review by an expert. The expert may be a healthcare professional or a domain-specific specialist that may have a deeper familiarity with the subject’s condition, or more experience related to the clinical context.

Based on the incremental evaluation of the one or more task-specific queries, a workup trajectory may be generated by the second AI technique. The workup trajectory may be configured by progressively refining the one or more actions resulting from each mapped trajectory of the plurality of mapped trajectories corresponding to the subset of knowledge bases. The refinement may be achieved through the application of first-order logics associated with the one or more actions. The first-order logics may evaluate which of the one or more actions may be appropriate based on the given condition of the subject. In some aspects, the workup trajectory may be derived from the predicted intermediate results of the result set by evaluating the first-order logics to recommend the one or more actions.

Based on the generated workup trajectory, a result may be displayed on the user interface accessible via the user endpoint. The result may comprise one or more refined actions, determined through the application of first-order logics. Each refined action indicated in the generated workup trajectory may further include a corresponding summary, generated by leveraging the second AI technique implemented by the LLE.

The second AI technique may be implemented using the LLM. In some aspects, the first AI technique applied during the development phase of the set of knowledge bases may be architecturally aligned with the second AI technique. By utilizing similar LLM capabilities in both development and execution phases, the LLE may effectively generate first-order logics and enable rule-based reasoning through consistent application of the first-order logics across the plurality of mapped trajectories, thereby enabling continuity, interpretability, and efficiency throughout a workflow.

In some aspects, a system is provided that includes one or more data processors and a non-transitory computer readable storage medium containing instructions which, when executed on the one or more data processors, cause the one or more data processors to perform part or all of one or more methods disclosed herein.

In some aspects, a computer-program product tangibly embodied in a non-transitory machine-readable storage medium, including instructions configured to cause one or more data processors to perform part or all of one or more methods or processes disclosed herein.

In some aspects, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by aspects and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 illustrates an overview of interacting with a large language expert (LLE) architecture to generate intelligent and customized outputs, in accordance with some aspects of the present disclosure.

FIG. 2 illustrates an exemplary block diagram, depicting development of a set of knowledge bases, stored in a knowledge repository, by leveraging a first AI technique, in accordance with some aspects of the present disclosure.

FIG. 3 illustrates an exemplary block diagram of a knowledge base (KB) server of the LLE architecture that interfaces with a second AI technique to generate personalized outputs for a subject based on an input dataset, in accordance with some aspects of the present disclosure.

FIG. 4 presents an illustration, depicting dynamic stacking mechanism through a hierarchical prioritization of a plurality of knowledge bases to access a subset of knowledge bases based on the input dataset, in accordance with some aspects of the present disclosure.

FIG. 5A shows an exemplary workflow overview of generating a result set comprising predicted intermediate results corresponding to the subset of knowledge bases, in accordance with some aspects of the present disclosure.

FIG. 5B shows an exemplary individual workflow that corresponds to the workflow overview, depicting a mapped trajectory of a plurality of mapped trajectories that corresponds to the subset of knowledge bases, in accordance with some aspects of the present disclosure.

FIG. 6 illustrates a first exemplary sequence diagram of generating the personalized outputs comprising the result set, and subsequently, summaries corresponding to a set of prompts, in accordance with some aspects of the present disclosure.

FIG. 7 illustrates a second exemplary sequence diagram, depicting enrichment of each predicted intermediate result of the result set based on a confidence metric, in accordance with some aspects of the present disclosure.

FIG. 8A presents a first illustration of a first exemplary graphical user interface (GUI), illustrating the result set derived from the input dataset or supplemental data associated with the subject, in accordance with some aspects of the present disclosure.

FIG. 8B presents a second illustration of the first exemplary GUI that enables a user to modify the illustrated result set as part of a review process, in accordance with some aspects of the present disclosure.

FIG. 9A presents a first illustration of a second exemplary GUI, depicting a recommended workup based on the result set shown in the first exemplary GUI, in accordance with some aspects of the present disclosure.

FIG. 9B shows a second illustration of the second exemplary GUI, enabling the user to modify the recommended workup shown in the first illustration of the second exemplary GUI, in accordance with some aspects of the present disclosure.

FIG. 10A illustrates a first set of metrics related to performance of the LLE architecture in determining decision factors, in accordance with some aspects of the present disclosure.

FIG. 10B illustrates a second set of metrics related to performance of the LLE architecture in identifying gaps in the workup recommendations, in accordance with some aspects of the present disclosure.

FIG. 11 illustrates an exemplary workflow of generating workup recommendations personalized in view of a given condition of the subject based on sequential and differential processing of a set of knowledge bases, in accordance with some aspects of the present disclosure.

FIG. 12 illustrates a simplified diagram of an example data processing network device or system for hosting the LLE architecture, in accordance with some aspects of the present disclosure.

### DETAILED DESCRIPTION

Some aspects of the present disclosure relate to techniques for generating personalized outputs based on processing of guidelines and supplemental data in view of a given condition of a subject. The disclosed techniques may be implemented using a large language expert (LLE) architecture, which may feature a hybrid artificial intelligence (AI) model. The hybrid AI model may synergistically integrate machine learning (ML) capabilities with deterministic, rule-based reasoning derived from rule-based logics indicated in the guidelines. By integrating the adaptability of the ML with defined structure of rule-based systems, the LLE architecture may offer a technical solution for applying complex, dynamic guidelines to real-world data.

The hybrid AI model may address technical problems inherent in traditional decision-support tools, which despite extensive investments, may have been proven difficult to build and may be impractical for clinicians to use. The challenges associated with traditional decision-support tools may be broadly categorized into two areas: first, the accurate and efficient encoding of clinical guidelines in a manner consistent with actual clinical practice; and second, practical implementation of such tools in clinical settings. Traditionally, the encoding of clinical guidelines may rely on either standalone ML models or rule-based systems. The ML models, such as large language models (LLMs), may be effective at capturing complex, probabilistic relationships within data, but since these models may often be implemented as black boxes, may be difficult to customize across institutions, and may require significant retraining to reflect updates in medical knowledge. Conversely, the rule-based systems, such as traditional expert systems, offer transparency and control; however they may be rigid, difficult to scale, and time-consuming to update or validate. When applied in isolation, both approaches may struggle to effectively translate the nuanced and sometimes ambiguous logic of clinical guidelines into actionable and accurate decision-making pathways.

In addition to the challenges associated with encoding clinical guidelines, the practical implementation of the traditional decision-support tools may encounter several obstacles. The obstacles may include: the presence of unstructured, inconsistent, or fragmented medical data across multiple platforms; the need to integrate clinical judgment based on subject’s medical data, as clinical cases may not often align neatly with predefined clinical guidelines; and the generation of results that lack sufficient explainability, which may undermine clinician trust. For instance, the ML models may generate plausible yet incorrect explanations, while rule-based systems may oversimplify complex clinical contexts. These challenges may hinder the effectiveness of traditional tools and contribute to their limited adoption by clinicians.

Some of the disclosed techniques may bridge the gap created by the technical problems, some of which are outlined herein by seamlessly integrating the adaptability and learning capabilities of the ML with the transparency and precision of rule-based reasoning. The implementation of the rule-based reasoning with the LLE architecture may be achieved by the integration of an LLM with rule-based logics encoded in the guidelines. The guidelines may be organized and deployed as knowledge bases by a knowledge base (KB) developer. The KB developer may be configured to develop each knowledge base by translating a guideline, which may codify an expert-guided workflow, into a structured format compatible with the LLM implemented within the LLE architecture. The expert-guided workflow may correspond to one or more actions that may be associated with a particular condition of the subject.

Therefore, each knowledge base may be developed to include the one or more actions corresponding to a particular condition. Each action of the one or more actions may further be linked to one or more variables that may inform or influence the action, and a rule-based logic defining conditions under which the action may be recommended. For instance, the rule-based logic may be encoded as conditional rules (e.g., if A and B, then recommend C), decision trees, or formal logic expressions (e.g., first-order logics). Therefore, the structured formats may enable the decision pathways, conditions, and variables to be explicitly defined and unambiguous, further enabling the LLM to interpret, apply, and reason over the encoded knowledge accurately.

The knowledge bases (or rule-based knowledge bases) may be developed by parsing the expert-guided workflow by leveraging a first AI technique, such as an LLM parser, implemented by the KB developer. However, an obstacle in managing rule-based or expert system knowledge bases stems from the challenge of converting a complex, interwoven, and interdependent set of expert rules into an executable code. The LLE architecture may mitigate such complexity by staying as close as possible to the original guidelines, leveraging English as an executable code. Additionally, the advanced reasoning capabilities of the LLM parser may enable accurate parsing of the logic embedded within the natural language of the guidelines, which may subsequently be translated into rule-based logics. The rule-based logics may then undergo an expert review process, maintaining the accuracy and relevance within the LLE architecture.

The knowledge bases, developed by the KB developer, may then be stored in a knowledge repository, from which a KB server may invoke one or more knowledge bases to support a given workflow at runtime. According to some aspects, the rule-based logic retrieved from an updated guideline may be used to update existing knowledge bases stored in the knowledge repository. The update process may include targeted modifications to the relevant natural language constructs within the knowledge bases. The updated knowledge bases may be captured as versioned artifacts, managed by a version manager that may have been deployed by the KB server. The versioned artifacts may be invoked by downstream applications powered by the LLE architecture in a configurable and context-aware manner. The use of versioned artifacts may provide a technical advantage by enabling clients healthcare institutions or organizations to adopt guideline updates according to their own timelines or schedules, supporting retrospective audits based on historical versions, and enabling compliance with clinical and regulatory standards.

However, updates in the LLE architecture may be performed independently, with experts modifying only the affected rules. The updates may form part of an update cycle that may offer a technical advantage by enabling focused changes without the need for full system reengineering. This decoupled update mechanism may reduce dependency on intermediary engineering teams, preserve the original intent of guideline authors, and significantly accelerate the deployment cycle.

The versioned artifacts, which may include a set of the knowledge bases adopted by the clients, may be used when a given workflow may be triggered. The workflow may be triggered in response to a request initiated by a user through the interface accessible via a user endpoint. The request may comprise an input dataset corresponding to a given condition of the subject. The input dataset may comprise the one or more of: a diagnosis, a potential diagnosis, a medical condition, or a corresponding code associated with the subject’s condition. In some aspects, the input dataset may further include supplemental data associated with the subject. The supplemental data may comprise additional clinical information that enhances the understanding of the subject’s condition. For instance, the supplemental data may include unstructured clinical notes, lab results, imaging reports, historical medical records, patient-reported information, etc.

The input dataset may be processed by the KB server that may be configured to access a subset of knowledge bases from a set of knowledge bases relevant to the input data set. The subset of knowledge bases may be accessed by: a) parsing the set of knowledge bases to retrieve a plurality of knowledge bases, and b) dynamically stacking the plurality of knowledge bases. In some aspects, the plurality of knowledge bases, that may have been dynamically stacked, may form the subset of knowledge bases. In some other aspects, the plurality of knowledge bases may be dynamically stacked based on the predefined levels of guideline authority, from which the subset of knowledge bases may be accessed.

For instance, a stacking engine associated with the KB server may invoke the plurality of knowledge bases by parsing the set of knowledge bases stored in the knowledge repository based on the given workflow. The invocation may include activating the one or more branching positions, corresponding to the one or more variables, that may inform or influence each action of the one or more actions defined in each knowledge base. The branching position may be activated based on the execution of task-specific queries on the input dataset, which may indicate the given condition of the subject.

In some aspects, the task-specific queries corresponding to the branching positions may be dynamically generated by leveraging a second AI technique such as the LLM that interfaces with the KB server. In some other aspects, the task-specific queries may be accessed from the set of knowledge bases and dynamically aligned with the input dataset to enable context-aware evaluation of the relevant variables, and corresponding relevant knowledge bases. The accessed task-specific queries may have been generated corresponding to the one or more variables by leveraging the first AI technique such as the LLM parser of the KB developer during the development of a knowledge base.

For instance, a task-specific query such as “Has the subject been diagnosed with colon cancer?” may have been accessed corresponding to a variable representing diagnosis. If the input dataset includes structured or unstructured data indicating a confirmed or suspected colon cancer diagnosis, the task-specific query may be dynamically aligned with that information, activating a corresponding branching position. The corresponding branching position may further evaluate the one or more nodes, each of which may represent a recommended action, or a related branching position. The evaluation of the related branching positions may depend on whether a given branching position may be activated upon the execution of the input dataset. The branching positions may be evaluated iteratively until all relevant conditions may be satisfied, resulting in a plurality of mapped trajectories associated with the activated branching positions.

The plurality of mapped trajectories may correspond to the plurality of knowledge bases, which may further be dynamically stacked based on the predefined levels of guideline authority. For instance, the plurality of knowledge bases may comprise open-source guidelines, institutional protocols, or department-level rules. The dynamic stacking mechanism may hierarchically prioritize department-level rules over institutional protocols, and institutional protocols over open-source guidelines. The hierarchical prioritization may offer a technical advantage by enabling clients to enforce localized clinical policies while maintaining alignment with broader standards. However, the hierarchical prioritization may also introduce a technical challenge, wherein higher-level rules may inadvertently override partially valid logic from lower-level rules, resulting in unintended behavior.

This technical challenge may be mitigated through the granular development of the knowledge bases, which may reduce discrepancies and enhance alignment across varying levels of guideline authority. For example, an open-source guideline may recommend both a mammogram and a breast MRI (magnetic resonance imaging) for a potential diagnosis of breast cancer. However, the client may adopt a more specific protocol that recommends only a breast MRI, considering the mammogram unnecessary in certain cases. Without sufficient granularity, a higher-priority guideline (e.g., an institutional guideline) may unintentionally override or inherit broader recommendations from a lower-priority guideline (e.g., an open-source guideline), resulting in conflicts such as introducing the mammogram that the institution may choose to deprioritize. Granular structuring of the knowledge bases may enable clear separation and precise inheritance of guideline elements, reducing the risk of inconsistencies during the dynamic stacking and execution mechanism.

Based on the dynamic stacking of the knowledge bases, the subset of knowledge bases may be accessed. The one or more task-specific queries relevant to each knowledge base of the subset of knowledge bases may be transmitted to a query generator. Upon accessing the task-specific queries, supplemental data associated with the subject may be accessed by the query generator from one or more data sources. The one or more data sources may include structured electronic health record (EHR) fields, clinical text notes, scanned documents, PDFs, or other heterogeneous medical artifacts, potentially comprising unstructured or inconsistent information. In some aspects, the supplemental data may generate structured subject records suitable for processing in conjunction with the subset of knowledge bases.

The query generator may generate a set of prompts based on the LLM prompt templates representing meticulously crafted structures that may contribute to the generation of precise and contextually relevant natural language queries. Based on the task-specific queries or the subject records derived from the supplemental data, a first set of prompts of the set of prompts may be generated. Each prompt of the first set of prompts may represent a task-specific query associated with the subset of knowledge bases, and the subject records. The first set of prompts may be incrementally evaluated by the second AI technique, i.e., the LLM implemented by the LLE architecture. The incremental evaluation may comprise executing the one or more task-specific queries of each mapped trajectory on the subject records. In some aspects, the one or more task-specific queries may also be executed on the input dataset.

Based on the incremental evaluation of the first set of prompts, a predicted intermediate result may be generated by the LLM for each task-specific query of the one or more task-specific queries. The predicted intermediate result may include: a) a response, comprising one of: “yes,” “no,” or “unknown,” or a string or integer bundle response, to each task-specific query; b) a description justifying the response; and c) one or more citations referencing the input dataset or the supplemental data associated with the subject.

The predicted intermediate result generated by the LLM may be transmitted to an inference router that may be configured to aggregate the predicted intermediate result of each task-specific query to generate a result set. The result set may then be output via the interface accessible through the user endpoint, enabling the user to conduct a review. During the review process, the user may override responses associated with the one or more predicted intermediate results of the result set.

The inference router may receive the overridden responses as feedback via the interface accessible through the user endpoint. The LLM may again be leveraged to update the corresponding predicted intermediate results based on the overrides. Updating each predicted intermediate result of the one or more predicted intermediate results may include: a) generating a revised justification aligned with the overridden response; and b) identifying one or more citations from the supplemental data or the input dataset that support the overridden response. In some aspects, the user may provide justification via the interface alongside the overridden response, which may then be incorporated as a citation linked to the predicted intermediate result.

According to some aspects, the inference router may transmit the result set to a scoring module, which may assign a confidence metric to each predicted intermediate result of the result set prior to outputting the result set on the interface. The confidence metric may be determined based on factors such as the accuracy, reliability, and relevance of the predicted intermediate result in the context of the supplemental data or the input dataset. In some aspects, the confidence metric may assist the user in prioritizing specific predicted intermediate results during the review process—wherein results with lower values corresponding to the confidence metric may warrant closer scrutiny compared to ones with higher values of the confidence metric.

During the review process, if the user may override a predicted intermediate result that may be assigned with a high confidence metric based on the subject records, the inference router may trigger additional processing. Specifically, the inference router may output overridden predicted intermediate result via an interface accessible through an expert endpoint for a secondary review. The expert may be a healthcare professional or a domain-specific specialist having a deeper familiarity with the subject’s condition, or more experience related to the clinical context.

Upon user review, the inference router may initiate a workup trajectory, progressively refining the one or more actions derived from each mapped trajectory within the subset of knowledge bases. The refinement may be achieved through the application of first-order logics associated with the one or more actions. The first-order logics may evaluate the one or more actions to determine an appropriate action based on the given condition of the subject and the predicted intermediate results of the result set.

Based on the generated workup trajectory, the refined one or more actions (or one or more recommended actions) may be transmitted to the query generator. The query generator may generate a second set of prompts of the set of prompts. Each prompt of the second set of prompts may represent an action or the one or more recommended actions. The second set of prompts may trigger the LLM to generate summaries of the recommended actions. The one or more recommended actions along with the summaries may be output as a result on the interface accessible through the user endpoint via the inference router.

The user may review the summaries, instructing the subject with a clear and concise recommended workup to complete prior to initiating treatment or consulting a specialist. Therefore, the LLE architecture, providing a hybrid approach, may not only facilitate efficient updates and customizations across healthcare institutions but also enable a more consistent and reliable application of guideline-driven care, ultimately enhancing clinical decision-making.

It will be appreciated that some embodiments disclosed herein may pertain to various non-medical use cases. For example, AI techniques may be used to generate task-specific queries and process data records in insurance workflows, regulatory-compliance workflows, or technical-support workflows, among others.

The disclosed techniques may offer significant technical advantages by effectively reconciling structured clinical guidelines with real-world data characterized by variable presence and confidence levels. The use of a modular knowledge base structure may enable seamless customization to accommodate diverse clinical settings. Interactive interfaces combined with confidence metrics enhance transparency, enabling users to understand and manage incomplete or conflicting data. Additionally, the integration of multiple AI techniques with rule-based logic may enable robust processing of both structured rules and unstructured, incomplete, or inconsistent data. This approach enables interpretable and flexible processing of the workflows, empowering users to interact dynamically with predicted decision points and building greater confidence in the clinical outcomes.

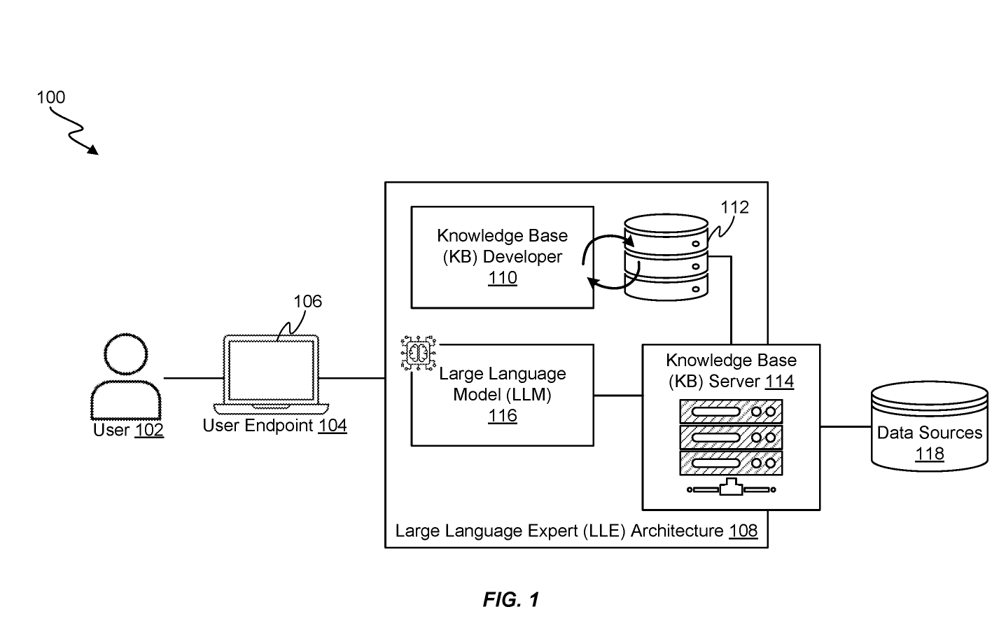

FIG. 1 illustrates an overview 100 of interacting with a large language expert (LLE) architecture 108 to generate intelligent and customized outputs, in accordance with some aspects of the present disclosure. The overview 100 represents a communication platform that leverages an artificial intelligence (AI) model referred to herein as a LLE, which may feature a hybrid architecture that synergistically combines large language model (LLM) capabilities with deterministic, rule-based reasoning. The hybrid architecture may strategically harness the contextual flexibility of LLMs while preserving precision and auditability of rule-based systems, enabling seamless interaction with both structured and unstructured data sources and supporting transparent, logic-driven decision-making.

According to some aspects, the hybrid architecture such as the LLE architecture 108 may be hosted on a cloud platform. The cloud platform may be a public, private, or hybrid cloud environment, depending on deployment requirements. In some other aspects, the LLE architecture 108 may be deployed on-premises within a secure enterprise infrastructure or offered as a managed service by a third-party provider. In some aspects, the LLE architecture 108 may be containerized such as using docker or similar container technologies to encapsulate the corresponding codebase, runtime, dependencies, and configurations into a portable execution environment. Containerizing the LLE architecture 108 may enable consistent deployment across diverse computing infrastructures (e.g., cloud platforms, on-premises servers, or edge devices) while also enhancing scalability, ease of maintenance, and system reliability.

A user 102 may access the LLE architecture 108 via an interface 106 accessible through a user endpoint 104. The interface 106 may be a dedicated interface specifically designed to facilitate interaction with the LLE architecture 108. In some aspects, the interface 106 may be implemented as a web-based application accessible through a public or private network. In some other aspects, the interface 106 may be deployed locally at the user endpoint 104 as a native application. The user endpoint 104 may include a desktop computer, a laptop, a tablet, a smartphone, a wearable device (e.g., smartwatch). A thin client terminal, or any other computing device capable of supporting access to the LLE architecture 108. The user endpoint 104 may operate locally or remotely, and may support access through web browsers, native applications, or virtualized environments.

The user 102, for example, a primary care physician or general practitioner—that may be a non-specialist unfamiliar with the subject’s case—may interact with the LLE architecture 108 by initiating a request via the interface 106. The request may include an input dataset corresponding to a subject, who may be experiencing a health condition and may need support in determining an appropriate workup trajectory. Given the growing complexity of diagnostic recommendations, users may face challenges in verifying that subjects have completed all required evaluations before consulting a specialist. The LLE architecture 108 may assist the users by analyzing the input dataset and generating structured guideline-aligned recommendations, thereby supporting timely and informed clinical decisions before specialist referral or initiation of a treatment.

The LLE architecture 108 may comprise a knowledge base (KB) developer 110, a knowledge repository 112, a knowledge base (KB) server 114, and a LLM 116. The KB developer 110 may initially be configured to develop the knowledge repository 112, which may further be used in determining the appropriate workup trajectory comprising the one or more workup recommendations. The KB developer 110 may translate clinical guidelines that codify expert-guided workflows into structured formats—a syntax compatible with the LLM 116. The structured formats may refer to formal logical representations of expert documents or guidelines that may be organized in a consistent and machine-readable way. The structured formats may be specifically designed to express clinical or domain-specific reasoning in a form that may be processed by the LLM 116. For instance, the logic may be encoded as conditional rules (e.g., if A and B, then recommend C), decision trees, or even formal logic expressions (e.g., first-order logics). Therefore, the structured formats may enable the decision pathways, conditions, and variables to be explicitly defined and unambiguous, enabling the LLM 116 to interpret, apply, and reason over the encoded knowledge accurately.

According to some aspects, the structured formats may also be aligned with natural language to preserve human readability and facilitate expert validation, thereby bridging the gap between formal computational logic and human understanding. The structured formats that encode logic, rules, and decision factors may be stored as individual, structured knowledge bases in the knowledge repository 112. The knowledge repository 112 may comprise a set of knowledge bases. In some aspects, each knowledge base of the set of knowledge bases may represent a distinct guideline, enabling a granular approach to customizing and managing content across one or more knowledge bases. The granular approach may facilitate streamlined updates, support version control, and enable incremental integration of new or revised guidelines. Moreover, the granular approach may enable evolving medical knowledge to be incorporated efficiently without disrupting existing workflows or affecting unrelated logic structures.

The one or more knowledge bases of the set, stored in the knowledge repository 112, may be utilized by the KB server 114. The KB server 114 may align the formal logic representations encoded in the one or more knowledge bases with prompt generation pipelines to dynamically compose prompts for the LLM 116. The queries may further be informed by subject-specific record data retrieved from the data sources 118. The data sources 118 may include structured electronic health record (EHR) fields, clinical text notes, scanned documents, PDFs, or other heterogeneous medical artifacts, potentially comprising unstructured or inconsistent information.

To support reliable reasoning, the KB server 114 may process such unstructured or inconsistent subject records into structured representations that map directly to the formal logic representations encoded in the one or more of knowledge bases. Notably, the translation process may be managed by the KB server 114 rather than the KB developer 110, supporting greater reusability and abstraction across various workflows. By offloading the translation process to the KB server 114, the LLE architecture 108 may flexibly adapt to heterogeneous and evolving data environments without the need for modifications to the underlying guideline representations stored in the knowledge repository 112.

The processed records may further enable subsequent generation of context-specific and customized outputs using the LLM 116. The LLM 116 may be implemented using models such as OpenAI’s GPT-4 Turbo, ChatGPT Enterprise, CoPilot (GitHub), or OpenAI’s o1 model, among others. These models may be capable of performing advanced natural language understanding, reasoning, and generation tasks, including semantic parsing, summarization, and context-aware response formulation. However, it will be appreciated that the models described herein are provided by way of example and without limiting the scope of the present disclosure. The LLM 116 may be updated or replaced with any suitable and more productive model that becomes available in the future.

Leveraging the capabilities of the LLM 116 whether current or subsequently upgraded in conjunction with the rule-based logics represented in each knowledge base, may facilitate automated generation of high-quality outputs. The outputs may include: diagnostic assessments, natural language explanations of the diagnostic assessments, tailored screening plans, pre-treatment workup recommendations, structured reasoning behind workup recommendations, human-interpretable summaries etc. Each output may be systematically aligned with established clinical and expert guidelines, as represented in the set of knowledge bases of the knowledge repository 112.

The outputs may be presented via the interface 106, where the user 102 may interact with the outputs in real time. The interaction may include reviewing, modifying, or overriding generated assessments, or workup recommendations based on clinical judgment or a new information. By enabling such a dynamic feedback loop, the overview 100 may support both automated decision-making and expert oversight. According to some aspects, feedback mechanism may further be leveraged to fine-tune and iteratively enhance artifacts of the LLE architecture 108 over time. The LLE architecture 108, through its tight integration of the LLM 116 with rule-based logic encoded in the knowledge repository 112, may offer a scalable, interpretable, and adaptable architecture for clinical decision support effectively addressing the complexity, variability, and evolving nature of real-world healthcare environments.

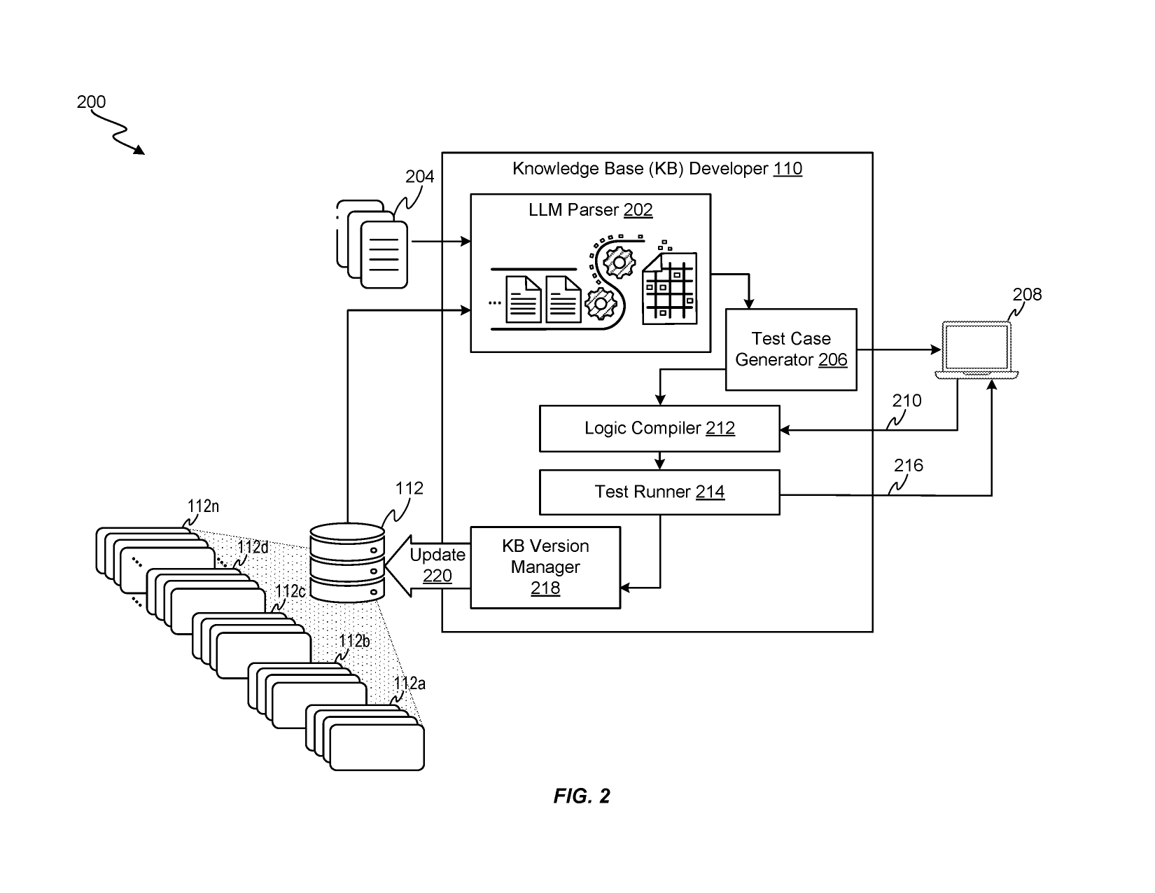

FIG. 2 illustrates an exemplary block diagram 200, depicting the development of a set of knowledge bases 112a-n, stored in the knowledge repository 112, by leveraging a first AI technique, in accordance with some aspects of the present disclosure. The development of the set of knowledge bases 112a-n may be a core mechanism that, in part, supports the hybrid LLE architecture. The LLE architecture 108 comprises the KB developer 110 that may be configured to translate documents 204 such as specific, versioned expert-authored documents into a declarative format, which may further be augmented for processing by the LLM 116.

The translation of the documents 204 comprising clinical guidelines may be facilitated by an LLM parser 202, which may serve as the first AI technique implemented by the LLE architecture 108 and operates as a component of the KB developer 110. The LLM parser 202 may utilize the natural language understanding capabilities of an LLM to interpret, extract, and restructure clinical information such as actions, variables indicating decision factors and conditional logics embedded within the documents 204. In some aspects, the documents 204, comprising guidelines or expert-authored protocols, may be automatically retrieved by the KB developer 110 from a variety of external sources. The external sources may include publicly available medical databases, web-based repositories of clinical guidelines (e.g., NIH, WHO, professional societies), subscription-based platforms, institutional knowledge libraries etc. The retrieval of the documents 204 may be facilitated through techniques including, for example, web scraping, API-based integration, or federated querying of structured data sources.

According to some aspects, retrieval-augmented generation (RAG) pipelines may be employed to dynamically identify and retrieve documents 204 that may be contextually relevant based on evolving inputs or domain-specific triggers. In some aspects, the LLM parser 202 may operate in conjunction with the RAG pipelines to enable real-time retrieval and contextual refinement of relevant guideline content. In some other aspects, the LLM parser 202 may be a part of a RAG architecture, enabling the LLM parser 202 to retrieve the documents 204 and translate the embedded guidelines into formal logic representations to develop knowledge bases and update the knowledge repository 112.

According to some aspects, the documents 204 may directly be provided to the KB developer 110 by one or more domain experts affiliated with a healthcare institution. In some aspects, the documents 204 may be contributed by experts or stakeholders associated with an organization responsible for developing or maintaining the LLE architecture 108. The documents 204 may be uploaded through secure data channels, document ingestion interfaces, or integrated content management systems. The resulting configuration may enable close alignment between institutional or organizational knowledge, and the structured logic derived for subsequent reasoning and decision support.

The retrieved or provided guidelines may subsequently be encoded into structured formats (i.e., formal logic representations) and stored as individual knowledge bases within the knowledge repository 112. The set of knowledge bases 112a–n (hereinafter referred to as knowledge bases 112a–n) may be systematically derived from the documents 204. Each knowledge base may be configured to encapsulate recommendations (e.g., one or more actions), one or more variables (e.g., decision factors) associated with the one or more actions, and rule-based logics that govern the one or more actions, all organized in a structured, machine-interpretable format.

However, a persistent challenge in the development of the knowledge bases 112a-n lies in converting complex, interdependent, and often ambiguous clinical rules into executable logic, a process that may traditionally require significant manual intervention and domain expertise. To address this, the LLE architecture 108 may bypass conventional complexity by preserving the original structure of the guidelines and leveraging English as an executable code. The disclosed approach may enable advanced language models to parse and reason over natural language representations directly, reducing the technical overhead of manual rule engineering while maintaining interpretability and accuracy to source material.

According to some aspects, the LLM parser 202 may utilize advanced language models such as OpenAI’s o1 model to accurately parse logic constructs embedded in guidelines and translate them into machine-interpretable formats suitable for subsequent reasoning. The structured representations may include first-order logic, decision trees, declarative rule sets, or rule graphs, each selected based on the nature and complexity of the underlying guideline. For instance, first-order logic may be used for representing granular logical dependencies, while decision trees may be used to capture high-level conditional workflows in a more interpretable structure. Furthermore, the declarative rule sets may be employed for encoding clinical assertions or eligibility checks in a concise and modular format.

Once translated, the structured representations may undergo expert review to validate clinical accuracy and ensure fidelity to the original guideline intent, balancing automation with human oversight and preserving trust in the resulting decision-support logic. To facilitate structured validation, a case generator 206 may be configured to retrieve the structured representations and automatically generate one or more test cases.

Each test case may represent a synthetic or real-world clinical scenario, comprising subject attributes, conditions, and contextual variables, designed to simulate the way the encoded logic may be applied in practice. The one or more test cases may enable the KB developer 110 to compile relevant subsets of guidelines or decision rules tailored to specific cases. For instance, if a patient presents with a known history of breast cancer, the case generator 206 may surface a targeted recommendation such as a diagnostic mammogram, aligning with established oncology workup protocols. The test cases may ensure that the logic accurately reflects real-world clinical pathways and supports both comprehensive and case-specific validation before integration into the knowledge repository 112. In some aspects, the one or more test cases may indicate identified ambiguities, contradictions, or gaps detected in the guideline content. The one or more test cases may then be presented via an interface accessible through an expert endpoint 208 for domain-specific experts to examine and refine the subsets of decision rules.

Following review, a modified case 210 may be transmitted to a logic evaluator 212 that may function as a validation component within the LLE architecture 108. Upon receiving the modified case 210 from the expert endpoint 208, the logic evaluator 212 may systematically analyze the revised logic against the original clinical rules and decision constructs. The logic evaluator 212 may check for logical consistency, completeness, and adherence to established guideline constraints. In some aspects, the logic evaluator 212 may identify any contradictions, missing conditions, or deviations from intended workflows, maintain both clinical accuracy and computational integrity of the domain-specific expert’s modifications. In some aspects, the logic evaluator 212 may automatically assess or validate the subsets of decision rules generated as test cases by the case generator 206.

Following the logic evaluation, a test runner 214 may execute the test case, which may be updated, in a controlled environment to simulate real-world application of the encoded logic. The test runner 214 may be configured to run the test case through predefined scenarios, validating output correctness, compliance with clinical protocols, and expected behavior across varied input parameters. The test runner 214 may assess whether the case produces accurate diagnostic recommendations, decision pathways, or alerts consistent with the clinical guidelines. Results from these simulations may be compiled and returned to the expert endpoint 208, providing actionable feedback 216 to further refine the logic embedded in the subsets of decision rules. In some aspects, the logic evaluator 212 alone may be sufficient to perform logic validation and test execution on the workup protocols, particularly in scenarios where limited variability or lower complexity may allow for streamlined assessment.

It will be appreciated that the logic evaluator 212 and the test runner 214 may work in tandem to iteratively refine the subsets of decision rules until predefined quality standards may be met for integration into the knowledge repository 112. The predefined quality standards may include alignment with current expert-approved guidelines, adherence to coherent and conflict-free logic, or compatibility with data schema of the knowledge repository 112.

The subsets of decision rules may then be used to develop one or more of the knowledge bases 112a–n. In some aspects, the subsets of decision rules may be used to update 220 the one or more of the existing knowledge bases present in the knowledge repository 112. The update(s) 220 to clinical guidelines may be initiated through targeted modifications to the relevant natural language constructs within the structured knowledge bases, as disclosed herein. Independently, domain-specific experts may revise only the affected rules within the LLE architecture 108, enabling focused changes without requiring full system reengineering. This decoupled update mechanism may reduce dependency on intermediary engineering teams, preserve the original intent of the guideline authors, and significantly accelerate deployment cycle.

Once the revisions are incorporated, the updated guidelines may be captured as a versioned artifact, which may be invoked by subsequent applications powered by the LLE architecture 108 in a configurable and context-aware manner. The versioned artifacts may enable healthcare institutions to adopt guideline updates on their own timelines, support retrospective audits based on historical versions, and enable compliance with clinical and regulatory standards. As a result, the LLE architecture 108 may retain its modularity, interpretability, and highly extensibility, even as medical knowledge evolves.

The creation and management of the versioned artifacts may be overseen by a KB version manager 218. The KB version manager 218 may be configured to track and store a comprehensive version history for each of the knowledge bases 112a–n within the knowledge repository 112. This capability may enable traceability, rollback to prior versions, and differential analysis across guideline iterations providing an infrastructure for regulatory compliance, institutional flexibility, and long-term auditability.

The versioning capabilities of the KB version manager 218 may also address practical challenges encountered in real-world healthcare settings that may often be difficult to manage in monolithic or static machine learning (ML) architectures. For instance, while guidelines are regularly updated, healthcare institutions often adopt these changes at different paces, depending on internal review processes, training schedules, and operational constraints. Version control may enable healthcare institutions to implement new protocols on their own timelines without disrupting legacy workflows.

In addition, versioned knowledge bases support auditability and retrospective analysis. For example, a healthcare institution may wish to retrospectively evaluate clinical decisions, measure compliance with historical guidelines, or investigate deviations from standard protocols. In such cases, referencing the specific version of the guideline may become essential that may have been active during the period under review. The KB version manager 218, therefore, enables robust support for audit trails, regulatory compliance, and quality assurance initiatives across time.

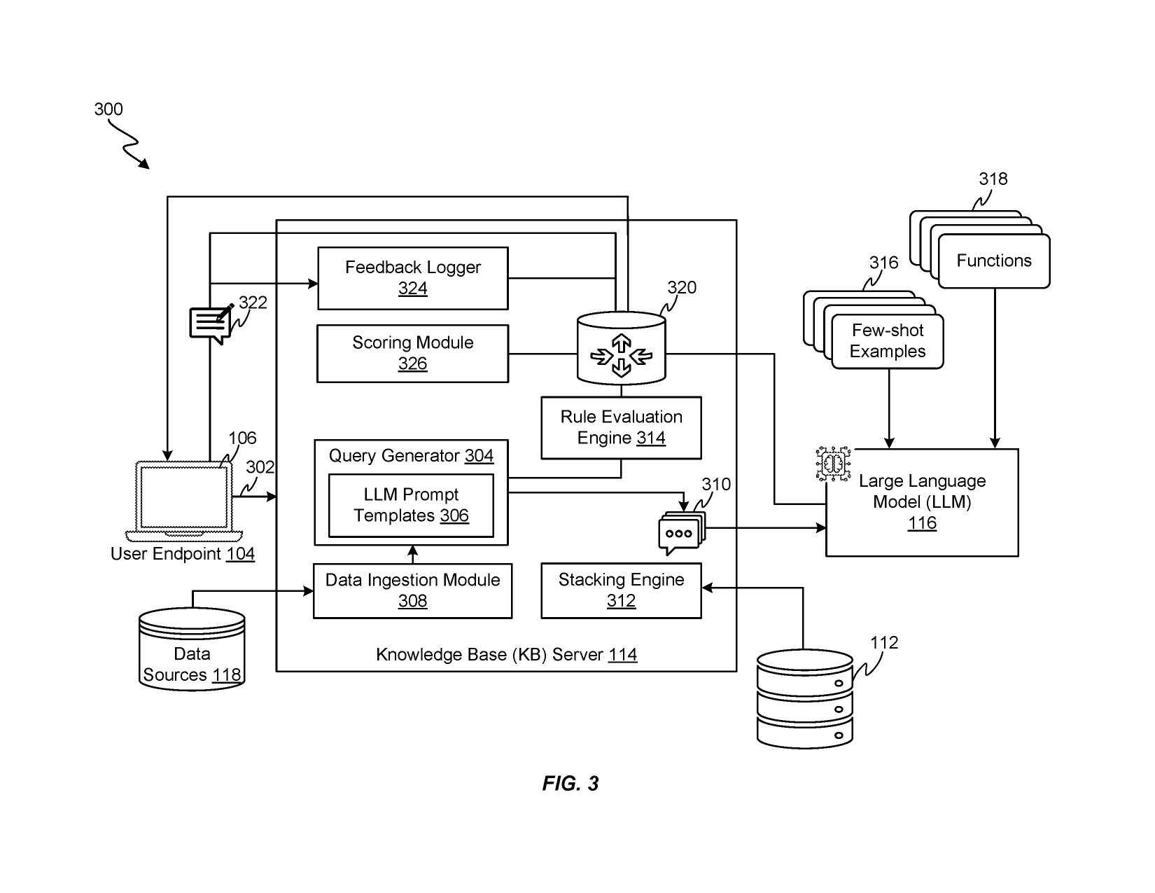

FIG. 3 illustrates an exemplary block diagram 300 of the KB server 114 of the LLE architecture 108 that interfaces with the second AI technique to generate personalized outputs for a subject based on an input dataset 302, in accordance with some aspects of the present disclosure. The KB server 114 may operate as a centralized infrastructure component within the LLE architecture 108, designed to manage and serve clinical knowledge efficiently. The KB server 114 may operate on a set of knowledge bases 112a-n that have already been versioned, organized, and curated reflecting specific guidelines adopted by a client (e.g., a healthcare institution). Building on such structured foundation, the KB server 114 may dynamically assemble, prioritize, and execute the one or more knowledge bases of the set of knowledge bases 112a-n at inference time to generate context-aware, institution-aligned clinical recommendations.

According to some aspects, rather than embedding clinical logic directly into individual applications, the KB server 114 may maintain and manage a dynamic stack of knowledge bases that may be assembled and prioritized based on the specific needs of a given application or clinical workflow. In some aspects, the dynamic stack of knowledge bases may correspond to a subset of knowledge bases from the set of knowledge bases 112a-n, which may be assembled and prioritized based on the given workflow. The subset of the knowledge bases may be accessed using a stacking engine 312. The stacking engine 312 may enable context-aware execution by selecting and layering relevant guidelines such as national standards, institutional protocols, or departmental rule according to predefined priority rules, enabling only the appropriate logic to be applied during inference.

The inference process may be initiated through a request submitted via the interface 106 accessible through the user endpoint 104. The request may include an input dataset 302 defined for a subject. In some aspects, the input dataset 302 may be defined for a subject to identify the one or more subject-specific diagnoses, symptoms, or characteristics. For example, the input dataset 302 may be defined to include a provisional or confirmed diagnosis, a description of a medical condition, or associated diagnostic codes. In some aspects, the input dataset 302 may further comprise supplemental data including laboratory test results, imaging findings, medication history, allergy information, demographic details (e.g., age, sex), or other clinically relevant health record data associated with the subject.

Based on the input dataset 302, the stacking engine 312 may access a plurality of knowledge bases from the set of knowledge bases 112a-n stored in the knowledge repository 112. Each knowledge base stored in the knowledge repository 112 may be developed to include: a) an action; b) one or more variables representing decision factors that determine applicability of the action; and c) a rule-based logic (e.g., a first-order logic) that specifies a condition under which the action may be recommended. The plurality of knowledge bases may be retrieved by parsing the knowledge bases 112a-n stored in the knowledge repository 112. Parsing each knowledge base may comprise evaluating one or more branching positions of the knowledge base, based on the execution of the one or more task-specific queries. The one or more task-specific queries may be questionable representations of the one or more variables, represented in the branching positions, in natural language format.

In some aspects, the one or more task-specific queries may be accessed from each knowledge base and may be dynamically aligned with the input dataset to retrieve the plurality of knowledge bases that may be relevant to the given condition of the subject. These one or more task-specific queries were generated by the first AI techniques such as the LLM parser 202 during the development of each knowledge base. In some other aspects, the one or more task-specific queries may be dynamically generated for the one or more variables associated with each knowledge base by leveraging a second AI technique, which may be implemented using the LLM 116. The stacking engine 312 may retrieve artifacts of each knowledge base, which may then be transmitted to a query generator 304. The query generator 304 may incrementally generate prompts for generating the one or more task-specific queries corresponding to the one or more variables. In some aspects, the one or more task-specific queries may be generated by the query generator 304 and executed on the input dataset 302 to access the plurality of knowledge bases.

Subsequently, the plurality of knowledge bases may be dynamically stacked by the stacking engine 312 based on predefined levels of guidelines authority. The dynamic stacking mechanism may hierarchically prioritize department-level rules over institutional protocols, and institutional protocols over open-source guidelines. The prioritization may enforce localized clinical policies resulting in the subset of knowledge base, while maintaining alignment with broader standards.

Upon accessing the subset of knowledge bases aligned with the given condition of the subject, the corresponding one or more task-specific queries associated with each knowledge base of the subset may be transmitted to the query generator 304. The one or more task-specific queries may further be executed on the supplemental data associated with the subject. The supplemental data may be retrieved from the data sources 118. In some aspects, the supplemental data may include part or all the EHR fields. In some aspects, the supplemental data may comprise unstructured or inconsistent records coming from sources including clinical text notes, scanned documents, PDFs, or other heterogeneous medical artifacts. The KB server 114 may facilitate the transformation of unstructured or inconsistently formatted records into structured representations that directly align with workflows—such as diagnostic assessments, screening recommendations, and pre-treatment planning—supported by the LLE architecture 108.

The transformation process may be facilitated by data ingestion module 308, which may be responsible for retrieving the supplemental data from the data sources 118 and converting subject records into formats compatible with the rule-based logics encoded in the knowledge bases. To achieve this, the data ingestion module 308 may leverage a combination of AI-powered natural language processing (NLP) techniques, entity recognition models, and structured data mapping algorithms. The following techniques may enable the data ingestion module 308 to retrieve clinically relevant information such as diagnoses, lab results, medications, and symptoms from diverse formats, including free-text clinical notes, scanned documents, PDFs, and structured EHR fields. The retrieved information may then be normalized against standardized vocabularies (e.g., SNOMED CT, ICD-10, LOINC) and aligned with the one or more variables or the rule-based logics specified in the knowledge bases. As a result, the transformed records may become queryable and interoperable, enabling accurate, context-aware reasoning during inference.

In some aspects, the transformed records may further be transmitted to the query generator 304. The query generator 304 may generate one or more sets of prompts 310 by utilizing LLM prompt templates 306. The LLM prompt templates 306 may represent meticulously crafted structures that may contribute to the generation of precise and contextually relevant natural language queries. By employing such templates, the query generator 304 may guarantee that the one or more sets of prompts 310 delivered to the LLM 116 may be both coherent and purpose-driven, enhancing the precision and dependability of the resulting outputs.

The one or more sets of prompts 310 may comprise a first set of prompts generated based on the one or more task-specific queries and the subject records (or transformed records). In some aspects, the first set of prompts may be generated based on the one or more variables specific to the subset of knowledge bases. Each prompt of the first set of prompts may represent a task-specific query associated with the subset of knowledge bases, and the subject records. In some aspects, the transformed records may be transmitted to the LLM 116 as a standalone prompt generated by the query generator 304. In some other aspects, the transformed records may be routed to the LLM 116 via the inference router 320.

The first set of prompts may be incrementally evaluated by the second AI technique such as the LLM 116. The incremental evaluation may comprise executing the one or more task-specific queries on the subject records. In some aspects, the one or more task-specific queries may also be executed on the input dataset. For each prompt of the first set of prompts, a predicted intermediate result may be generated including: a) a response such as “yes,” “no,” or “unknown,” or a string or integer bundle response; b) an explanation justifying the response; and c) one or more citations from the subject records that the LLM 116 utilized in forming the reasoning.

The predicted intermediate results corresponding to the first set of prompts may be aggregated to generate a result set. The aggregation of the predicted intermediate results may be performed by the inference router 320. The result set may then be transmitted to the interface 106 via the inference router 320 for review by the user 102. To build trust and enhance explainability, the responses corresponding to the one or more task-specific queries may be presented as a condensed list, enabling the user 102 to easily inspect the underlying reasoning and cited portions of the subject record.

Presenting the result set in such clear and accessible format may reduce the effort and potential errors associated with interpreting complex health records. By explicitly exposing variables alongside explanations and citations, the LLE architecture 108 may enhance transparency and empower users to quickly identify and correct any inaccuracies or ambiguities. The proactive correction may further prevent errors from propagating to subsequent processing workflows. Additionally, presenting decision factors that may indicate the predicted intermediate results using a terminology familiar to the users may enable the LLE architecture 108 to align its outputs with real-world clinical practice, fostering greater trust and enhancing the explainability of AI-generated predictions.

According to some aspects, confidence metrics may further be computed against each response generated by the LLM 116. The response generated by the LLM 116 may be transmitted to a scoring module 326 via the inference router 320. The scoring module 326 may enrich each result with a confidence metric expressed as a numerical value or a categorical label (e.g., high, medium, or low confidence). To compute the confidence metrics, the scoring module 326 may leverage techniques including: native confidence indicators provided directly by an LLM API; logit or probability scores associated with the generated tokens (when accessible); or post-hoc calibration methods such as temperature scaling to adjust confidence estimates. In some aspects, entropy measurements of the generated responses may be used to assess uncertainty. In some aspects, consistency checks may be performed by re-prompting the LLM 116 and comparing answers for stability. In some aspects, self-reflection prompting techniques may be employed to prompt the LLM 116 to assess its own confidence in the response. It will be appreciated that these techniques may be used individually or in combination to generate reliable confidence metrics for the responses produced by the LLM 116. However, the techniques described herein are not intended to limit the scope of the present disclosure.

The confidence metrics generated by the scoring module 326 may directly influence the interface 106 accessible through the user endpoint 104. The confidence metrics may enable the user 102 to prioritize the variables based on associated confidence levels. For example, responses for which the computed confidence metric exhibits a relatively low value, may be flagged for the user’s immediate attention over high confidence ones. During a review process, the user 102 may override responses to one or more predicted intermediate results, which may be received as feedback 322 by the inference router 320. The overridden responses may be received via the interface accessible through the user endpoint. The LLM 116 may then be leveraged to update the corresponding predicted intermediate results based on the overridden responses. Updating each predicted intermediate result of the one or more predicted intermediate results may include: a) generating a revised justification aligned with the overridden response; and b) identifying one or more citations from the supplemental data or the input dataset that support the overridden response.

In some aspects, a justification may be provided by the user via the interface 106 along with the overridden response, and may be incorporated as a citation to the predicted intermediate result. In some aspects, an expert may also be prompted to review outputs exhibiting low values of the confidence metric alongside the user 102. According to some aspects, if the user 102 overrides a predicted intermediate result associated with a high confidence metric, the inference router 320 may route the overridden result to an interface accessible via the expert endpoint 208 for a secondary review.

Once the result set is reviewed by the user 102, the KB server 114 may proceed to evaluate the actions corresponding to the variables—across which the predicted intermediate results were generated—by leveraging a rule evaluation engine 314. The rule evaluation engine 314 may receive the predicted intermediate results directly from the LLM 116 or as the feedback 322 from the interface 106 via the inference router 320. The rule evaluation engine 314 may refine the actions retrieved from the subset of knowledge bases by applying the first-order logic associated with each action, in conjunction with the predicted intermediate results. Since first-order logic formulas are deterministic, the first-order logics may be systematically evaluated to determine the applicability of each action.

To enhance explainability, the LLM 116 may be employed to generate summaries articulating a reasoning for recommending a particular action. The summaries may be generated for each recommendation based on a second set of prompts of the one or more sets of prompts 310 that may be generated by the query generator 304. The summaries may be human-readable comprising explanations based on the subject record, the associated variables, the corresponding first-order logic expressions, and the predicted intermediate results. Further, the summaries may be combined with detailed reasoning and the one or more citations for each underlying variable, offering significantly greater transparency and interpretability compared to traditional rule-based systems.

The summaries, along with the result sets comprising outputs in response to the task-specific queries, may collectively be referred to as the outputs of the LLM 116. In some aspects, such outputs may be generated by the LLM 116 using few-shot examples 316. The few-shot examples 316 may refer to a prompt engineering technique where the LLM 116 may be provided with a limited number of representative input-output pairs as part of its prompt. The few-shot examples 316 may serve as contextual demonstrations that guide the model in understanding how to perform a given task (e.g., extracting clinical information, generating explanations, etc.) without the need for extensive retraining.

In some other aspects, the outputs of the LLM 116 may be generated based on functions 318. The functions 318 may represent specialized tools, procedures, or callable modules integrated with the LLM 116, which may allow the model to delegate certain tasks such as arithmetic calculations, date comparisons, structured lookups, or accessing external data to deterministic software components. These function-enabled prompts may enhance the reliability and precision of the LLM's outputs, particularly in areas where language models typically underperform, such as numeric reasoning or structured data retrieval. The use of the functions 318 may enable the LLE architecture 108 to combine the natural language understanding capabilities of the LLM with the robustness of traditional rule-based computation.

Further, the actions indicating a recommended workup for the given subject, along with corresponding summaries generated by the LLM 116, may be output on the interface 106 for user review and refinement. This configuration may support interactive decision-making, while allowing the user oversight to remain integral to the recommendation workflow.

By building the application atop the LLE architecture 108, a modular intervention experience may have been introduced, offering several technical advantages. The application—accessible as the interface 106 associated with the LLE architecture 108—may enable user (or expert) intervention at the level of variables or decision factors, enabling targeted corrections before errors propagate through downstream logic. This fine-grained control not only prevents error compounding but also enhances long-term system reliability, as such corrections may be incorporated into future updates to the knowledge bases. Additionally, the rule-based and deterministic nature of the application may enhance interpretability and explainability, rationalizing the intervention process. The user 102 may easily identify and manually correct known limitations of the LLM 116—such as issues with date calculations or numerical reasoning—without disrupting the broader clinical logic or workflow.

Furthermore, the LLE architecture 108 may leverage function-enabled LLM components to support precise and targeted interventions. The modular configuration may enable builders to quickly isolate failure modes, apply patches, test, and redeploy changes without requiring a full system overhaul—improving maintainability and accelerating iteration cycles. To further enhance adaptability, a feedback logger 324 may be employed to capture the feedback 322 (e.g., corrections to the predicted intermediate results or modifications to the recommended workup). The feedback 322 may inform future updates to the knowledge bases and underlying logic, reinforcing the LLE architecture's robustness and suitability for real-world applications. While not explicitly depicted in the figure, various components or modules of the KB server 114 may be functionally connected or interact with one another to facilitate seamless execution of the described operations.

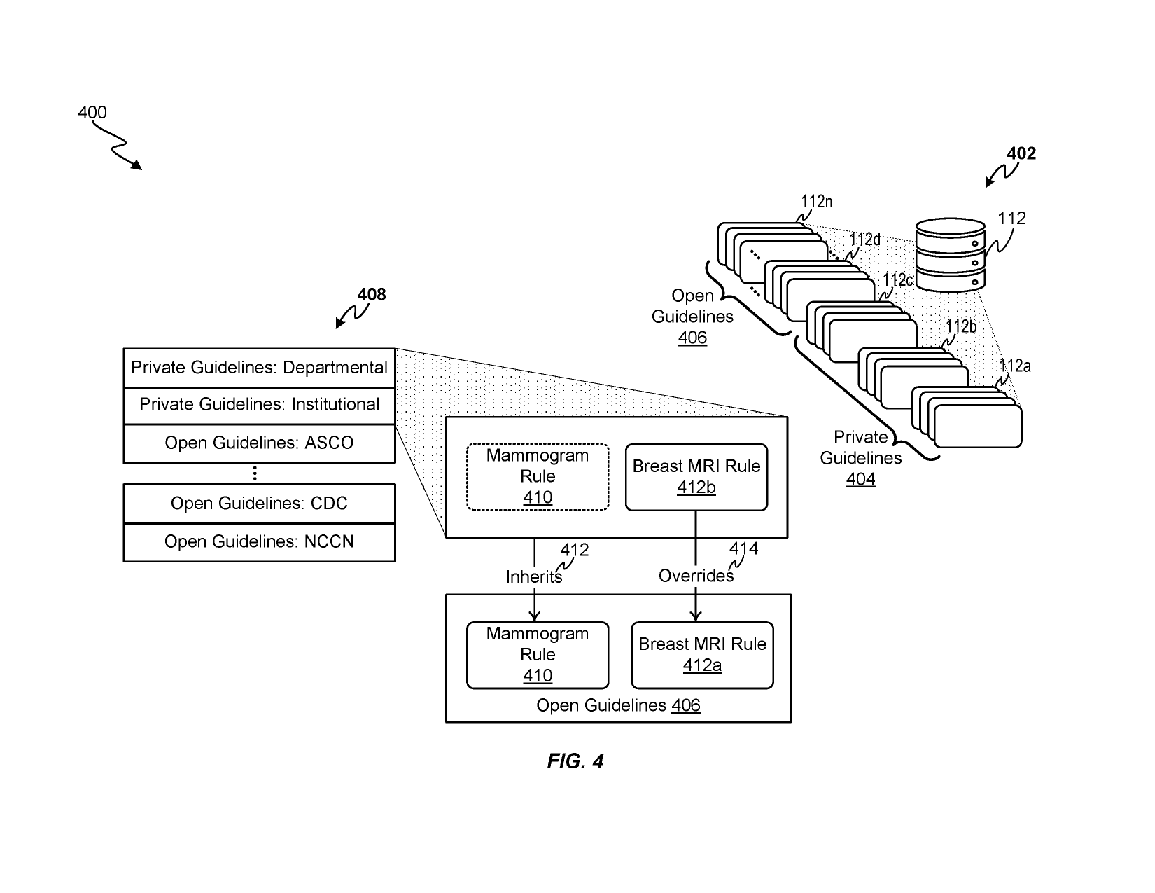

FIG. 4 presents an illustration 400, depicting dynamic stacking mechanism through hierarchical prioritization of the knowledge bases 112a–n to access the subset of knowledge bases based on the input dataset 302, in accordance with some aspects of the present disclosure. At 402, the knowledge repository 112 illustrated comprises the set of knowledge bases 112a–n, which may be categorized into private guidelines 404 and open guidelines 406. As illustrated, knowledge bases 112a–c may correspond to the private guidelines 404, while knowledge bases 112d–n may represent the open guidelines 406. However, the specific allocation of knowledge bases to either category is not fixed; any number of knowledge bases within the knowledge repository 112 may be designated as private or open, depending on access restrictions, source ownership, or institutional policies.

As disclosed herein, each clinical guideline or each of the documents 204 may be encoded as an individual knowledge base within the knowledge repository 112. Structuring the knowledge repository 112 in such a manner may enable modular access and further facilitate subject-specific customization of recommendations based on the input dataset 302. The modular access may also support flexible prioritization, illustrated at 408, enabling the stacking engine 312 to evaluate and rank relevant knowledge bases (e.g., the plurality of knowledge bases) based on contextual relevance, institutional preferences, or subject-specific factors.

After parsing the set of knowledge bases 112a-n, the plurality of knowledge bases relevant to the given condition of the subject may be retrieved. The plurality of knowledge bases may then be stacked dynamically based on predefined levels of guidelines authority, as illustrated at 408. The stacking of the plurality of knowledge bases may aggregate guidelines from multiple sources, drawing recommendations from both the open guidelines 406 and the private guidelines 404.

Widely recognized open guidelines such as the national comprehensive cancer network (NCCN), centers for disease control and prevention (CDC), and American society of clinical oncology (ASCO) may typically be assigned foundational priority levels. While the open guidelines 406 establish broadly accepted clinical standards, they may be assigned to lower priority relative to private guidelines 404 that may be tailored to the institution. Private guidelines 404 may include departmental-level protocols, which often take precedence over broader institutional guidelines due to their specialized focus. Consequently, the stacking engine 312 may hierarchically prioritize knowledge bases by first considering private departmental guidelines, followed by institutional guidelines, and finally open guidelines 406, thereby allowing relevant and specific protocols to guide clinical decision-making.

For instance, if conflicting actions are identified between the open guidelines 406 and the private guidelines 404, the applicable rule may be selected from the knowledge base assigned the highest priority. In some aspects, the conflicting actions may instead be surfaced to the user 102 for review and decision-making. Therefore, the hierarchical prioritization supports the implementation of layered clinical policies. The hierarchical prioritization may result in the subset of knowledge bases tailored to the input dataset and context, ensuring that only the relevant guidelines may be invoked during clinical decision-making.

As illustrated at 408, the open guidelines 406 include a mammogram rule 410 and a breast MRI rule 412a, which may correspond to a broader category, such as cancer. In contrast, the private guidelines 404 may represent rules associated with a particular condition, such as breast cancer. The private guidelines 404 inherits 412 the mammogram rule 410 from the open guidelines 406 while including their own breast MRI rule 412b. In such a case, the breast MRI rule 412b in the private guidelines overrides 414 the corresponding breast MRI rule 412a from the open guidelines. The inheritance and override mechanism may enable private guidelines 404 to adopt broadly accepted recommendations, such as mammograms, while customizing or superseding specific protocols including breast MRI, to better fit institutional preferences or requirements.

However, the hierarchical prioritizing process may introduce a possibility of unintentional overrides, where higher-priority rules partially or fully supersede logic from lower-priority sources, potentially leading to unexpected outcomes. This may be analogous to variable shadowing or overloading in programming languages. To mitigate unintended side effects from rule conflicts, the knowledge bases 112a-n may be maintained with high granularity—for example, cancer-type-specific bases such as breast cancer rather than a broad oncology base—thereby limiting the scope of interaction between rules.

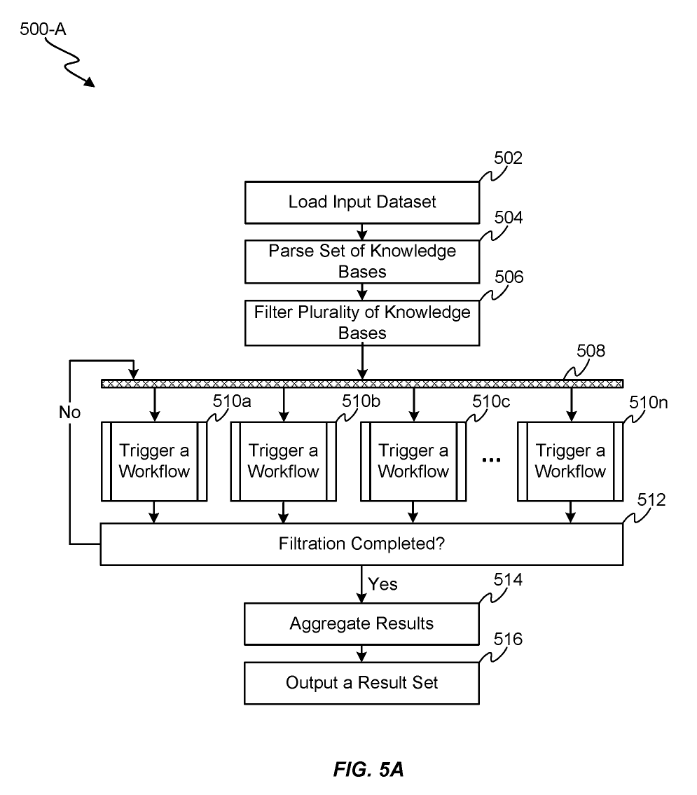

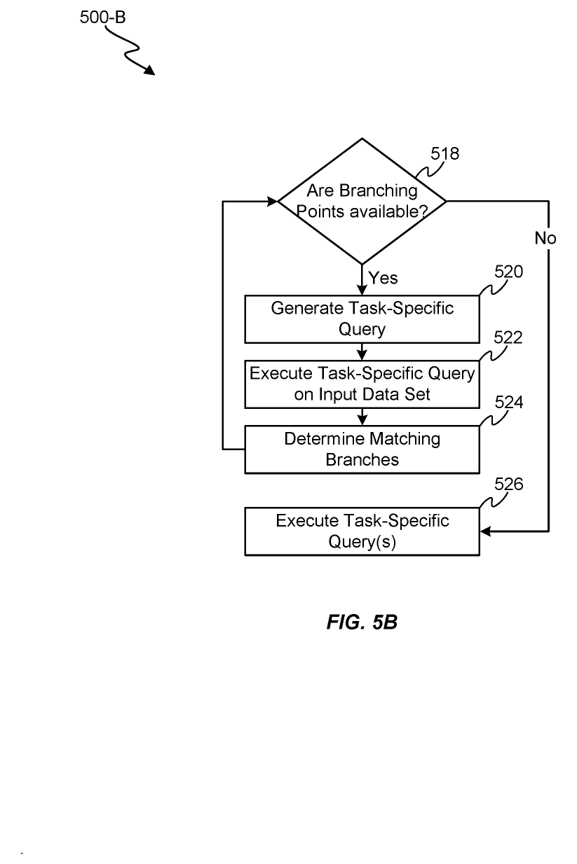

FIG. 5A shows an exemplary workflow overview 500-A of generating a result set comprising predicted intermediate results corresponding to the subset of knowledge bases, in accordance with some aspects of the present disclosure. The subset of knowledge bases may be accessed based on the hierarchical prioritization of the plurality of knowledge bases, as illustrated in FIG. 4. However, the exemplary workflow overview 500-A may depict retrieval process of the plurality of knowledge bases from the set of knowledge bases 112a-n, which may further be stacked dynamically to access relevant guidelines based on the hierarchical prioritization. The exemplary workflow overview 500-A further depicts the result set generated based on the accessed relevant guidelines.

At block 502, the input dataset 302—received via the interface 106 associated with the LLE architecture 108 accessible through the user endpoint 104—may be loaded into the KB server 114. The input dataset 302 may include a provisional or confirmed diagnosis, a description of the medical condition, associated diagnostic codes, or other supplemental data relevant to the subject.

At block 504, the stacking engine 312 may parse the set of knowledge bases 112a-n stored within the knowledge bases 112a-n of the knowledge repository 112. Parsing the set of knowledge bases 112a-n may correspond to identifying the guidelines that may be consistent with the given condition of the subject, based on the provided input dataset. Each knowledge base of the set 112a–n may be assessed based on branching positions, which may be activated by task-specific queries.

At 506, a plurality of knowledge bases determined to be consistent with the condition of the subject may be filtered from the set of knowledge bases 112a-n. The filtration process may include multiple individual workflows (at block 510a-n), where each knowledge base within the plurality of knowledge bases may be evaluated individually. In some aspects, the individual workflow processes may be performed in series, for example, such as if a particular workflow requires input from a previous workflow process. For example, certain clinical guidelines may be intertwined where the execution of one guideline inherently triggers or depends on another. In some aspects, the individual workflows may be performed in parallel, for example, if the separate workflow processes do not rely on a result from another workflow process that may be performed simultaneously. Additionally, individual workflows may be started and completed without regard to other workflows that may be operating. At block 512, upon a completion of at least one workflow of the individual workflows, the filtration may be evaluated to determine whether the filtration is completed.

If additional processing is required, the process may return to synchronizer 508 for appropriate queuing. If no additional processing is required, results of the individual workflows may be forwarded as appropriate. Results of the individual workflows may comprise the predicted intermediate results that may be generated based on guidelines such as a subset of mapped trajectories corresponding to the subset of knowledge bases that pertain to the given condition of the subject. The subset of mapped trajectories may be the plurality of mapped trajectories corresponding to the plurality of knowledge bases, based on the knowledge bases that were developed at granular level. In some aspects, the subset of mapped trajectories may be retrieved from the plurality of mapped trajectories, where the subset may present the guidelines from the highest priority levels.

At block 514, the forwarded results such as the predicted intermediate results, indicated based on activated branching positions from the individual workflows, may be aggregated into a result set. The predicted intermediate results may correspond to the variables (or decision factors) that determine the applicability of recommended actions based on the given condition of the subject. At block 516, the result set may be output on the interface 106.

FIG. 5B shows an exemplary individual workflow 500-B that corresponds to the workflow overview 500-A, depicting a mapped trajectory of the plurality of mapped trajectories that corresponds to the subset of knowledge bases, in accordance with some aspects of the present disclosure. The individual workflow 500-B of the individual workflows (at blocks 512a-n) may be used to identify whether a guideline in the set of knowledge bases 112a-n may correspond to a diagnosis or one or more symptoms indicated in the input dataset 302.

At block 518, a query may be configured to determine whether a branching position is available. At block 520, based on a branching position, a task-specific query may be generated to configure the corresponding trajectory. As illustrated, the task-specific query may be dynamically generated for a variable associated with the branching position by leveraging the second AI technique implemented using the LLM 116. For example, a task-specific query may be generated to evaluate whether the subject has been diagnosed with colon cancer. At block 522, the task-specific query may be executed on the input dataset 302, and based on the result, a corresponding trajectory may be activated.

In some aspects, the task-specific query corresponding to a variable may be accessed from the knowledge base associated with the guideline being processed. During the development phase, the knowledge base may be constructed using the first AI technique to define variables that determine the applicability of the recommended action(s) specified within that knowledge base. Each variable may represent a decision factor comprising a variable name and an associated task-specific query. These task-specific queries may be accessed and dynamically aligned with the input dataset based on the given condition of the subject, enabling context-aware evaluation of the relevant variables. For example, a task-specific query such as “Has the subject been diagnosed with colon cancer?” may have been accessed corresponding to a variable representing diagnosis. If the input dataset includes structured or unstructured data indicating a confirmed or suspected colon cancer diagnosis, the task-specific query may be dynamically aligned with that information, activating a branching position or corresponding trajectory.

At block 524, one or more matching branches may be identified based on an activated trajectory path. In some aspects, the matching branches may correspond to one or more nodes representing recommended actions based on the activated branching position. For example, if a patient is diagnosed with cancer and is over 40 years old, a guideline indicating a mammogram may be retrieved as part of the matching branch logic. In some aspects, the matching branches may correspond to one or more variables, which may require further evaluation against the input dataset 302. The evaluation may be performed by returning to block 518, enabling a dynamic reassessment of the subject’s clinical context based on the one or more variables.

The branching position in the guideline may indicate that the trajectory is to proceed toward a particular node based on the one or more variables (or the task-specific queries) that pertain to the input dataset 302. For instance, if a subject is over 40 years old and has blood pressure exceeding 130/80 mmHg, the trajectory may follow one path; otherwise, the trajectory may proceed along an alternative path. In some aspects, the task-specific query may focus on a single variable to configure the trajectory, as disclosed. This granularity may enable more precise alignment between the subject’s specific clinical context and the guideline logic, thereby enhancing accuracy and relevance of the resulting recommendations.

The individual workflow 500-B may be iteratively executed until a mapped trajectory is fully resolved, for example, logic associated with the trajectory has been processed to a conclusive outcome, thereby, contributing to a subset of mapped trajectories that collectively represent the subject’s clinical pathway. At block 526, if no further branching positions are identified, the workflow may proceed to execute one or more task-specific queries derived from the filtration of the plurality of knowledge bases on the subject records accessed through the data sources 118. The execution of the task-specific queries on the subject records may yield responses that validate the decision factors corresponding to the subject. In some aspects, the execution may also generate responses to one or more task-specific queries that may not have been explicitly defined in the input dataset 302 initially provided for the subject.

If no branching positions may be found to be relevant based on the input dataset 302, the corresponding individual workflow may be conditionally exempted from execution. The exemption may occur when none of the decision factors or task-specific queries are associated with a given knowledge base align with the subject’s clinical context, eliminating the need for further evaluation within that workflow. As a result, only workflows with actionable relevance contribute to the subset of mapped trajectories, enhancing computational efficiency and ensuring that recommended actions remain focused and contextually appropriate.

The blocks in the exemplary workflow overview 500-A and the exemplary individual workflow 500-B are illustrated in a specific order, while the order may be modified, for example, some blocks may be performed before others, and some blocks may be performed simultaneously. The block may be performed by hardware, software, or a combination thereof.

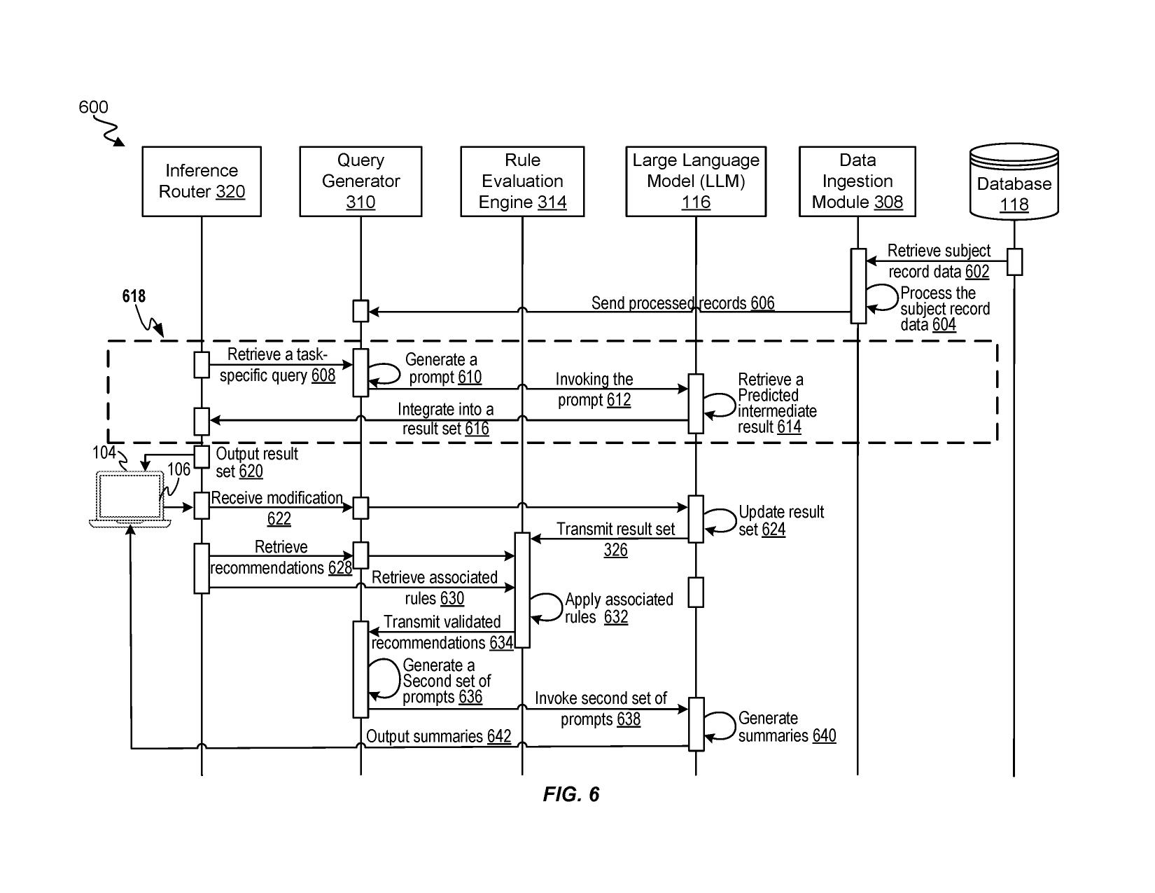

FIG. 6 illustrates a first exemplary sequence diagram 600 of generating the personalized outputs comprising the result set and, subsequently, summaries corresponding to the one or more sets of prompts, in accordance with some aspects of the present disclosure. The first exemplary sequence diagram 600 may be based on one or more task-specific queries, corresponding to the variables, that may be derived through the subset of mapped trajectories, as detailed in the exemplary individual workflow 500-B. At 618, the task-specific queries may be executed on the subject records to generate the predicted intermediate results.

At block 602, the data ingestion module 308 may retrieve the supplemental data or the subject records from the data sources 118. At block 604, the subject records comprising unstructured and potentially inconsistent information from one or more sources may be processed. Processing the subject records may include compiling inputs from multiple sources and converting unstructured information into a structured format suitable for downstream analysis. In some aspects, the data ingestion module 308 may employ AI-powered techniques to identify and resolve inconsistencies within the subject’s records. For instance, the module may infer missing values, normalize conflicting data entries, or reconcile duplicate or semantically similar fields.

At block 606, the processed records may be transmitted to the query generator 304. At block 608, the query generator 304 may retrieve a task-specific query from the inference router 320. In some aspects, the one or more task-specific queries may be retrieved in bulk by the query generator 304. At block 610, a prompt corresponding to the retrieved task-specific queries and the processed records may be generated by the query generator 304 using the LLM prompt templates 306.

At 612, the prompt may invoke the LLM 116. At 614, a predicted intermediate result may be generated by the LLM 116 by processing the prompt comprising the task-specific query and the processed records of the subject. In some aspects, the task-specific query corresponding to the decision factor may be transmitted directly to the LLM 116, and the predicted intermediate result may be generated by executing the task-specific query on the processed records. The predicted intermediate result may comprise: a) a response such as “yes,” “no,” or “unknown,” or a string or integer bundle response; b) an explanation justifying the response; and c) one or more citations from the subject records that the LLM 116 utilized in forming the reasoning.

At 616, the predicted intermediate result may be transmitted to the inference router 320 to be integrated with other predicted intermediate results. The integration of the results may generate a result set. In some aspects, the processes at 618, may correspond to the execution of the one or more task-specific queries on the subject records based on a first set of prompts of the one or more sets of prompts. The processes, at 618, may be iteratively repeated for each task-specific query of the one or more task-specific queries. The iteration may continue until all the decision factors may have been addressed.

At 620, the result set may be output on the interface 106 corresponding to the LLE architecture 108, accessible through the user endpoint 104. At block 622, a modification to one or more predicted intermediate results in the result set may be received via the interface 106. The modification may include altering responses associated with the one or more predicted intermediate results based on the user’s clinical judgment, domain expertise, or contextual knowledge of the subject. For instance, a user may update a response of a variable if a particular decision factor is determined to be irrelevant or absent in the subject's case, or if latest information has become available that was not initially captured in the input dataset. In some aspects, the user 102 may adjust the predicted intermediate results in response to the reasoning or rationale provided by the LLM 116 such as clarifications, supporting evidence, or logic trails thereby enabling human-in-the-loop refinement. This capability may support flexibility and clinician oversight, ensuring that the final recommendations remain clinically appropriate and aligned with the given condition of the subject.

At block 624, the LLM 116 may update the one or more results in the result set. Updating the one or more results may comprise modifying or regenerating explanations in response to changes made to one or more responses by the user 102. The re-generation may include re-evaluating the underlying reasoning, adjusting contextual interpretations, and generating updated natural language outputs that reflect the modified clinical scenario. In some aspects, the update process may also include identifying and presenting appropriate supporting citations, clinical evidence, or guidelines relevant to the revised inputs, thereby maintaining the accuracy, traceability, and clinical validity of the recommendations.

The updated result set may also be synchronized across the inference router 320 and, in some aspects, may additionally be output via the interface 106. However, the processes at blocks 622, 624, and 626 may be optional, as in some aspects, no modifications may be received via the interface 106 accessible through the user endpoint 104.

At block 628, recommended actions originally derived alongside the variables may be retrieved by the rule evaluation engine 314 from the inference router 320. In some aspects, the recommended actions may instead be re-retrieved directly from the guidelines if an update to the result set has resulted in a significant modification to the clinical scenario. At block 630, the rules associated with the recommendations may also be retrieved by the rule evaluation engine 314. In some aspects, the recommended actions and the associated rules may be concurrently retrieved by the rule evaluation engine 314.

At block 632, the associated rules corresponding to the potential recommendations may be applied by the rule evaluation engine 314. The rules may be expressed as first-order logic expressions, which may be evaluated over one or more variables applicable for the given condition of the subject. As first-order logic formulas are deterministic, the rules may be systematically evaluated to determine the applicability of each recommendation. At block 634, applicable actions as determined by the rules may be transmitted to the query generator 304.

At 636, the query generator may generate a second set of prompts of the one or more sets of prompts. The second set of prompts may invoke the LLM 116 to generate summaries for each applicable recommended action, at 638. At 640, the summaries for each applicable recommended action may be generated by the LLM 116 based on the second set of prompts. The summaries may include an explanation of why a particular action may have been recommended, referencing the one or more variables that contributed to its applicability. Additionally, each summary may incorporate the one or more citations (e.g., clinical guidelines, or evidence sources), thereby enhancing the interpretability, traceability, and clinical validity of the recommendation. At 642, the summaries may be output on the interface 106 of the LLE architecture 108.

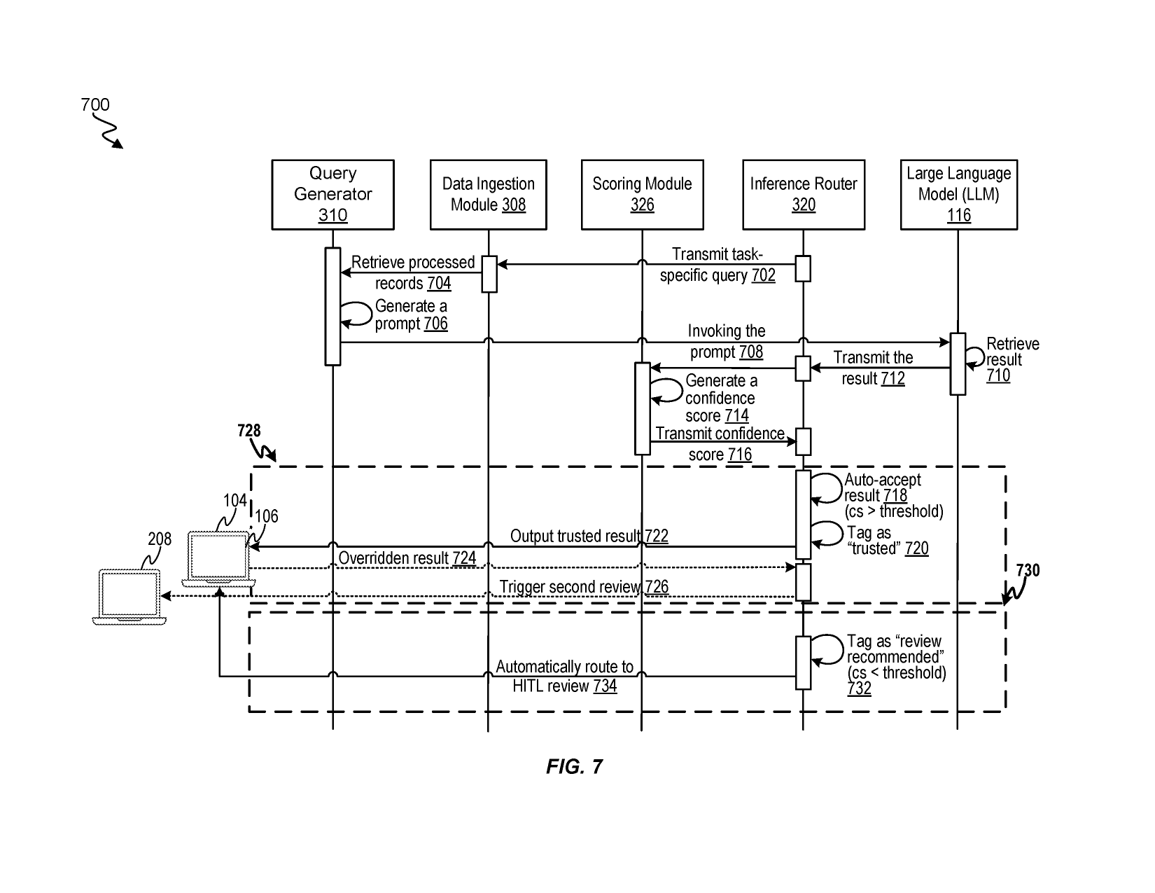

FIG. 7 illustrates a second exemplary sequence diagram 700, depicting enrichment of each predicted intermediate result of the result set based on a confidence metric, in accordance with some aspects of the present disclosure. The enrichment of predicted intermediate results with confidence metrics may offer a technical advantage by enabling the output displayed on interface 106 to guide user 102 in prioritizing review based on assigned confidence levels. The confidence metrics may enable the user 102 to efficiently focus on decision factors that require immediate attention, with low-confidence responses being prominently flagged. In some aspects, expert intervention may be integrated by prompting a clinical expert to review low-confidence outputs, or high-confidence outputs that may have been overridden by the user 102, thereby reinforcing accuracy, and reliability in the decision-making process.

At block 702, a task-specific query may be transmitted by the inference router 320. At block 704, the processed records of the subject may be retrieved by the query generator 304 from the data ingestion module 308. The retrieval of the processed records may be triggered by the transmission of the task-specific query. In some aspects, retrieval of the processed records may not be required each time a task-specific query may be received particularly when multiple task-specific queries associated with the same subject may have to be executed, thereby enhancing performance of the LLE architecture 108 and reducing redundant data access.

At block 706, a prompt may be generated corresponding to the processed records and the task-specific query. At block 708, the prompt may be transmitted to the LLM 116 for processing. At block 710, a predicted intermediate result corresponding to the prompt may be generated by the LLM 116. The result may include a response (e.g., yes, no, or unknown, or a string or integer bundle response) to the task-specific query, an explanation supporting the generated answer, and one or more citations from the subject’s processed records that substantiate the reasoning behind the result.

At block 712, the result may be transmitted to the scoring module 326 via the inference router 320. At block 714, the scoring module may generate a confidence score corresponding to the result. In some aspects, the confidence score may be specifically generated for the response within the predicted intermediate result, as the response serves as the initial deciding factor in determining the relevance or applicability of the associated recommendation. The confidence score may reflect the strength, completeness, or clarity of the supporting evidence drawn from the subject’s processed records.

At block 716, the confidence score may be transmitted to the inference router 320. At block 718, the inference router 320 may automatically accept a result if the confidence score meets or exceeds a predefined threshold. In some aspects, when a predicted intermediate result is accepted by the inference router 320, then the corresponding variable may not be presented for user review on the interface 106, thereby rationalizing the review process. However, in some other aspects, user 102 review may still be required depending on system configuration or contextual relevance. At block 720, predicted intermediate results with a confidence score equal to or above the predefined threshold may be programmatically tagged as “trusted” by the inference router 320. Following the classification, the inference router 320 may proceed to output the result, along with the corresponding tag, to the interface 106 for further use or visibility, at block 722.

At block 724, feedback may be received via the interface 106, accessible through the user endpoint 104. The feedback may include a modification to the predicted intermediate result, such as the result being manually overridden by the user 102. At block 726, a second review may be triggered by the inference router if the result, tagged as trusted, is overridden by the user 102. In some aspects, the second review may be conducted by the user 102. In some other aspects, the predicted intermediate result may be forwarded to an expert via the expert endpoint 208 for secondary evaluation. The expert may be a healthcare professional or a domain-specific specialist. In some aspects, the expert may also be the healthcare provider to whom the subject may be assigned for care.

Furthermore, if the confidence score associated with the predicted intermediate result falls below the predefined threshold, the result may be routed through the processes defined at 730. At block 732, such a predicted intermediate result may be tagged as “review recommended.” Subsequently, at block 734, the predicted intermediate result may be output on the interface 106 for review by the user 102.

The processes at 730 (e.g., blocks 732 and 734) may differ from the processes at 728, representing an alternative evaluation or escalation path specifically designed for low-confidence outputs. The distinction between processing flows reflects a resilient and adaptive system architecture—capable of modulating its inference strategy based on confidence levels, thereby strengthening both the reliability and clinical safety of the decision-making process.

While the second exemplary sequence diagram 700 illustrates the processing of a single task-specific query, the same procedural logic may be systematically applied across all remaining task-specific queries associated with the subject. Each individual predicted intermediate result may undergo confidence evaluation and contextual enrichment before being aggregated by the inference router 320. This aggregation phase ensures that a comprehensive, context-aware result set may be delivered to the interface 106—enabling users to engage with an output that may be both clinically grounded and intelligently prioritized.

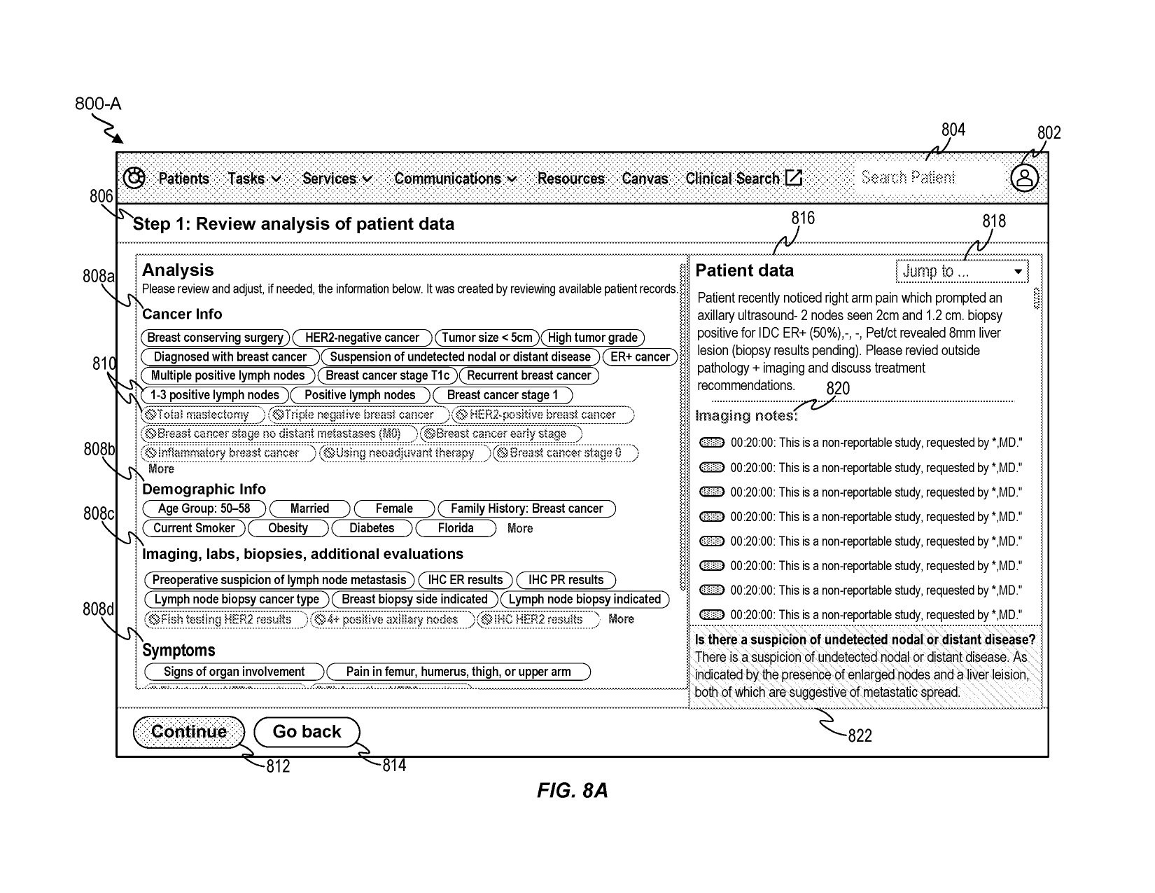

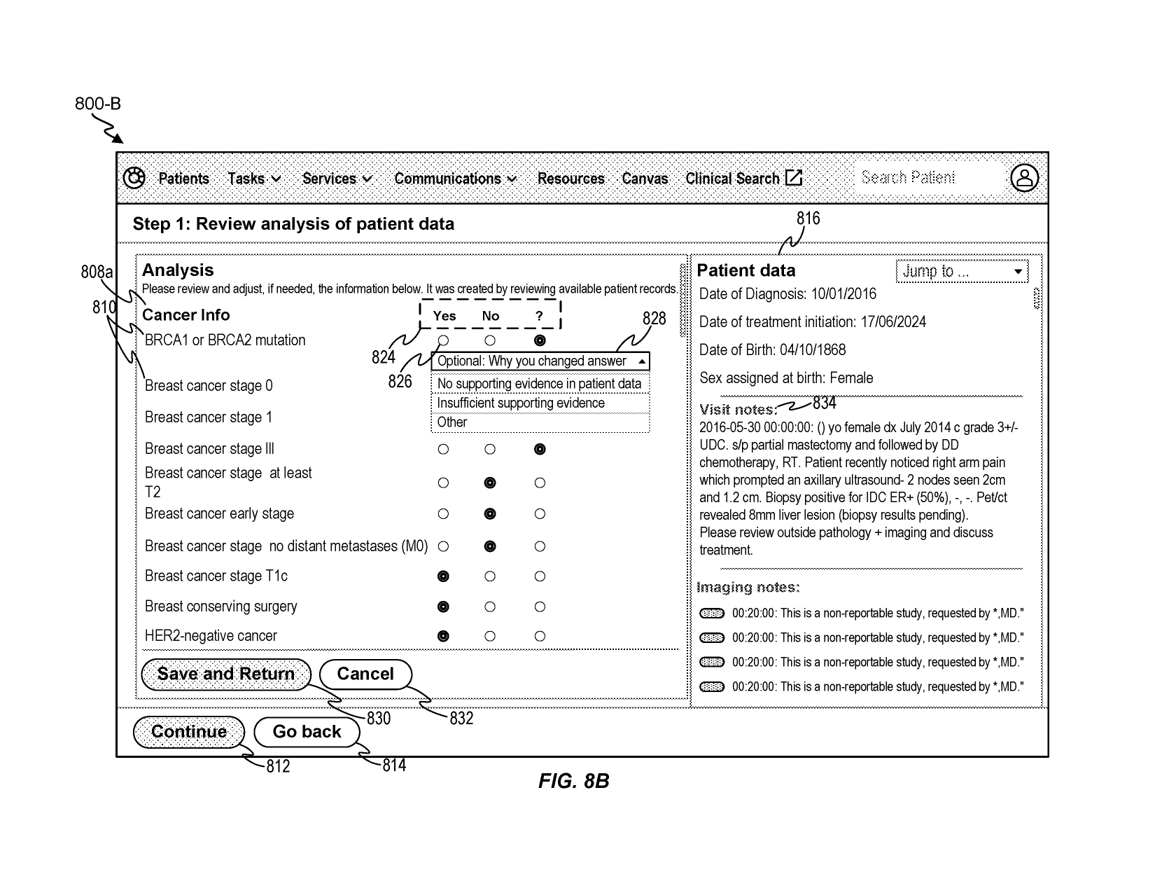

FIG. 8A presents a first illustration of a first exemplary graphical user interface (GUI) 800-A, illustrating a result set derived from the input dataset or supplemental data associated with a subject, in accordance with some aspects of the present disclosure. The illustrated result set may comprise one or more predicted intermediate results 810, each derived from the execution of one or more task-specific queries related to the variables. The one or more predicted intermediate results 810 may be determined using the second AI technique, which leverages the LLM 116 to generate responses based on the one or more task-specific queries.

The one or more predicted intermediate results 810 may be accessible based on an input dataset defined by a user 102. The input dataset may comprise a diagnosis, a potential diagnosis, a medical condition, or a corresponding code associated with the given circumstance of the subject. In some aspects, the input dataset may comprise supplemental data associated with the subject. The input dataset may be defined by the user 102 after signing into a secure profile 802, which not only provides access to personalized subject information but also ensures the privacy and integrity of sensitive medical data. The secure profile 802 may offer the user 102 controlled access to a range of subjects, facilitating a rationalized, individualized approach to data management and decision-making. The user 102 may search for information related to any subject within the range of subjects through a search field 804.

The one or more predicted intermediate results may be displayed on the first exemplary GUI, representing an analysis 806 of the subject data that may be derived from the input dataset or the supplemental data accessed through the data sources 118. The analysis 806 may encompass several distinct sections, each dedicated to a specific aspect of the subject data. The sections may include: a first section 808a, which presents information pertinent to the subject's specific circumstance (e.g., cancer diagnosis); a second section 808b, which outlines demographic details of the subject; a third section 808c, which provides evaluations such as imaging, biopsy results, laboratory data, and other relevant diagnostic information; a fourth section 808d, that highlights potential symptoms associated with the subject; and additional sections as necessary to present a comprehensive view of the subject's condition. The first section 808a, the second section 808b, the third section 808c, the fourth section 808d, and so on, may be collectively referred to as section 808.

Each section 808 may incorporate one or more predicted intermediate results 810. The predicted intermediate results may include outcomes that may be present in the subject based on the execution of one or more task-specific queries applied to the input dataset or supplemental data associated with the subject. For example, the task-specific queries may include: "Does the patient have positive lymph nodes?", "Is there a preoperative suspicion of lymph node metastasis (e.g., identified through imaging or palpable nodes on examination)?", or "Are there abnormalities observed on computed tomography (CT) or MRI scans that are considered suspicious but inconclusive for metastases?" Based on responses to such task-specific queries, the variables or decision factors may either align with or diverge from the subject's specific circumstance. The variables either align with or diverge from the subject's specific circumstance may result in the one or more predicted intermediate results 810, which may be reviewed by the user 102.

Upon reviewing the analysis 806, the user 102 may proceed with further processing by selecting a continue button 812. Alternatively, the user 102 may opt to revise the input dataset by selecting a go back button 814, which may navigate the user 102 to the input dataset entry interface or, in some instances, direct the user 102 to the home page of the interface for broader navigation.

The first exemplary GUI may further present a concise view of the subject data 816, offering a high-level summary of the subject’s condition. The subject data 816 may further comprise imaging notes 820 documenting the chronology of diagnostic studies conducted. The imaging notes 820, which may include timestamps and details about the requesting physician, enable the user 102 to track the progression of subject diagnostics. From the subject data 816, the user 102 may seamlessly jump to additional sections via a jump field 818, gaining access to additional information, advanced analyses, or clinical recommendations that support informed decision-making. Each of these additional sections may also provide a high-level summary reflective of the subject’s evolving condition.

The first illustration of the first exemplary GUI 800-A may also present a query 822, prompting the user 102 to evaluate the suspicion of undetected nodal or distant disease. The query 822 invites the user 102 to assess the potential for metastatic spread, as suggested by the presence of enlarged nodes and a liver lesion, both of which may indicate possible distant metastasis. The query 822 may serve as an interactive decision point, enabling the user 102 to determine whether further diagnostic evaluation or clinical action may be required based on the subject's current condition.

FIG. 8B presents a second illustration of the first exemplary GUI 800-B that enables the user 102 to modify the illustrated result set as part of a review process, in accordance with some aspects of the present disclosure. Modifying the result set, as depicted in the first illustration of the first exemplary GUI 800-A, may comprise adjusting the one or more predicted intermediate results 810 during the review process. In some aspects, the user 102 may initiate the review process by selecting one or more sections 808, which, in turn, triggers the second illustration of the first exemplary GUI 800-B. In some aspects, the continue button 812 may result in the second illustration of the first exemplary GUI 800-B.

The second illustration may display the one or more predicted intermediate results 810 corresponding to a list of decision factors. Each decision factor of the list may be mapped to one of the responses 824 (e.g., “yes,” “no,” or “unknown,” or a string or integer bundle response), which may constitute part of an intermediate result, further including reasoning and one or more supporting citations for the corresponding response. The user 102 may view the reasoning and one or more supporting citations by clicking on the corresponding decision factor. The user 102 may modify the one or more predicted intermediate results by changing the responses 824 associated with the decision factors. The selection may be facilitated through interactive radio buttons 826 that the user 102 may click to change the corresponding responses.

Based on a change to a response associated with one of the decision factors, a field 828 may appear, presenting one or more options in a drop-down menu to explain the reason for the change. The user 102 may select one of the options to provide an explanation for the modification made to the decision factor. The explanation may serve as feedback for the LLE 108 or the LLM 116, enabling the models to be fine-tuned accordingly.

The modification to the predicted intermediate results may be saved through a save and return button 830, which may preserve the changes and returns the user 102 to the first illustration of the first exemplary GUI 800-A. Alternatively, the user 102 may cancel the modifications by clicking a cancel button 832, which may return the first illustration of the first exemplary GUI 800-A. In some aspects, the cancel button 832 may be used to return to the first illustration if no changes have been made to the predicted intermediate results.

Further, the subject data 816, presented in the second illustration of the first exemplary GUI 800-B, may include details such as a date of diagnosis, date when treatment may have been initiated, the subject’s date of birth, sex, and other pertinent factors. The subject data 816 may also include visit notes 834, which capture the subject's clinical history from the date of diagnosis through to the present, providing a comprehensive record of ongoing care and medical evaluations. The visit notes 834 may be configured to provide the user 102 with a high-level summary, enabling for efficient review, and further enabling the user 102 to make informed adjustments to the predicted intermediate results 810, as necessary.

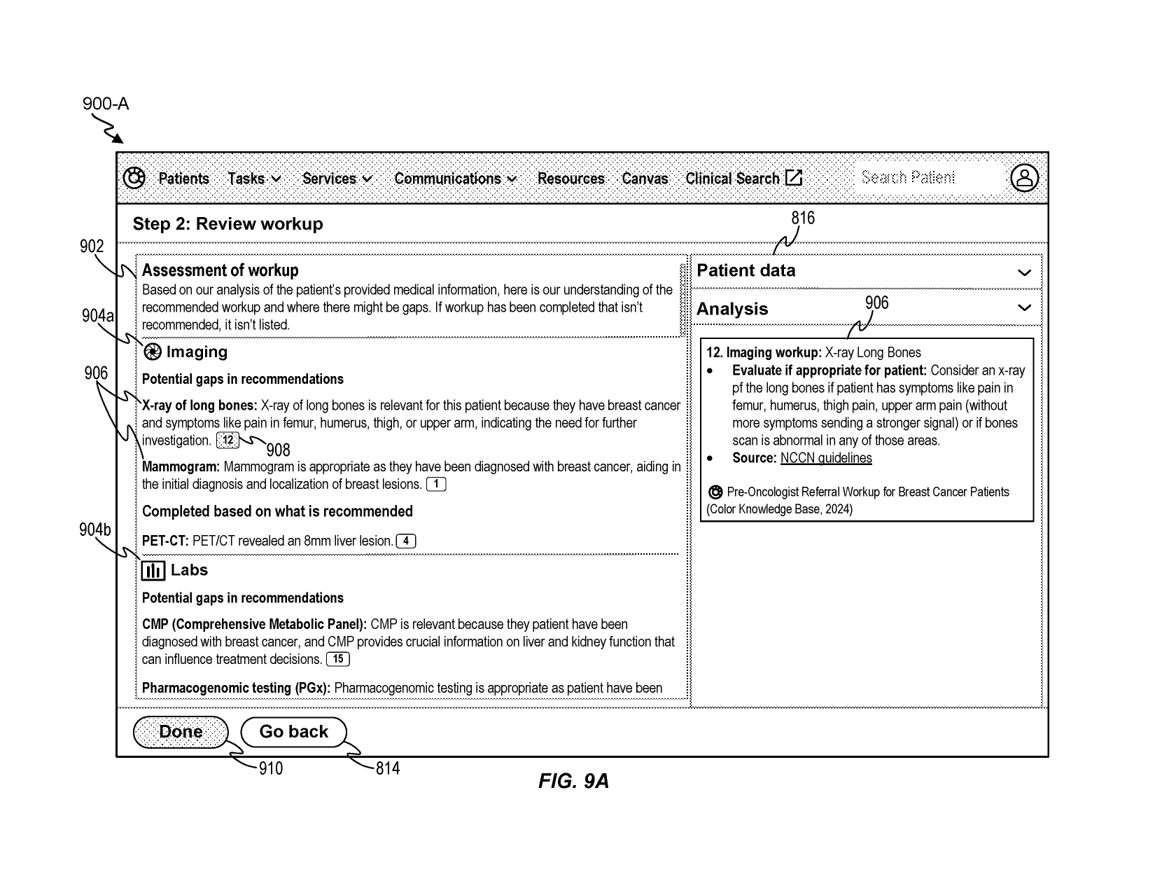

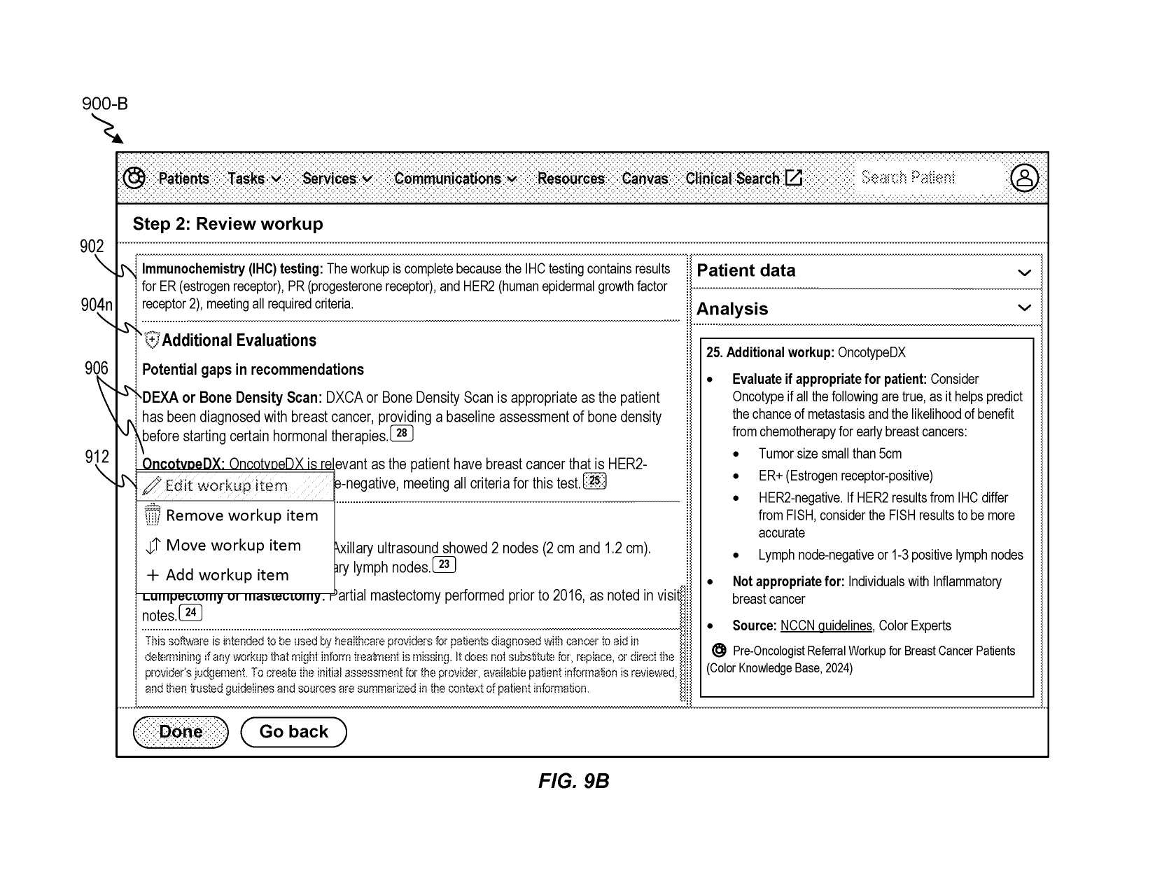

FIG. 9A presents a first illustration of a second exemplary GUI 900-A, depicting a recommended workup 902 based on the result set shown in the first exemplary GUI, in accordance with some aspects of the present disclosure. The recommended workup 902 may comprise one or more workup items 906 indicating actions suggested in view of the subject’s circumstances and the one or more predicted intermediate results. In some aspects, the recommended workup may highlight potential gaps in the recommendations, reflecting actions that may not have been yet completed. Additionally, any completed workups that may not align with the current recommended plan, may be deliberately let off from the display, thereby ensuring that only the recommended actions and pending execution may be prominently highlighted for review.

The recommended workup 902 may include the one or more workup items 906, organized under structured categories such as imaging 904a, laboratory tests 904b, …, or other additional evaluations. Each workup item may further be categorized as either completed or pending (indicating a gap), with associated explanations and supporting information drawn from the subset of knowledge bases to clarify the rationale behind the recommendation. For instance, in the case of imaging, the user 102 may be presented with a list of necessary imaging studies, noting whether they have been completed, are still pending, or have been identified as necessary for further evaluation.

Each workup item of the one or more workup items 906 may be associated with a reference number, such as reference number 908 exemplified by the X-ray of long bones recommendation. The reference number 908 may serve as a unique identifier, enabling the user 102 to quickly cross-reference the specific knowledge base or guideline related to the recommended action, as illustrated on the right side of the second exemplary GUI 900-A. By clicking on the reference number, the user 102 gains direct access to the relevant clinical guidelines, research evidence, or expert recommendations that underpin the action. This feature may enhance the transparency of the decision-making process, enabling the user 102 to review the rationale behind each recommendation and assess its alignment with the current clinical standards.

The user 102 may edit, add, move, or remove one or more workup items 906 as needed during the review process. Once the review is complete, the user 102 may click on a done button 910, indicating that the recommended workup 902 may have been thoroughly reviewed and may be ready to be finalized or forwarded for further action. Alternatively, the user 102 may return to the first exemplary GUI by clicking the go back button 814, enabling the user 102 to revisit previous sections or adjust, as necessary.

FIG. 9B shows a second illustration of the second exemplary GUI 900-B, enabling the user 102 to modify the recommended workup 902 shown in the first illustration of the second exemplary GUI 900-B, in accordance with some aspects of the present disclosure. The modification of the recommended workup 902 may comprise modifying one or more workup items 906. The one or more workup items 906 may be under one or more structured categories such as imaging 904a, laboratory tests 904b, …, or other additional evaluations 904n.

The user 102 may select a specific workup item to modify. Upon selection, an options tab 912 may appear, offering the following actions: a) edit workup item, b) remove workup item, c) move workup item, or d) add workup item. The options may enable the user 102 to customize the workup items 906 as needed to align with the subject’s evolving condition, clinical guidelines, or personal preferences. For instance, the user 102 may modify an existing item if current information has emerged or if a different course of action is required. Alternatively, the user 102 may choose to remove workup items 906 that may no longer be relevant or add new ones based on further assessments. This flexibility may ensure that the recommended workup 902 may be tailored to the individual subject’s needs, providing a dynamic and user-responsive interface for efficient clinical decision-making.

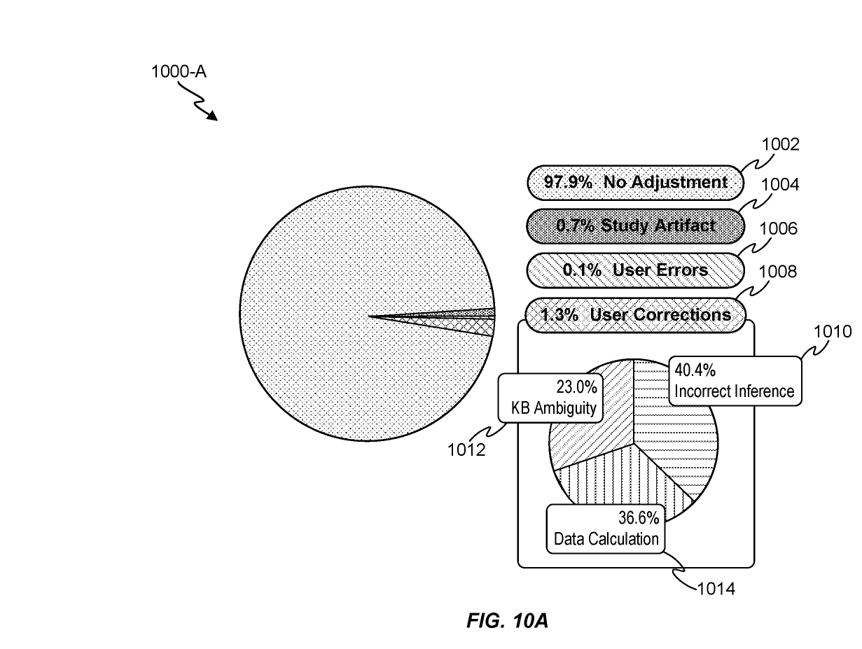

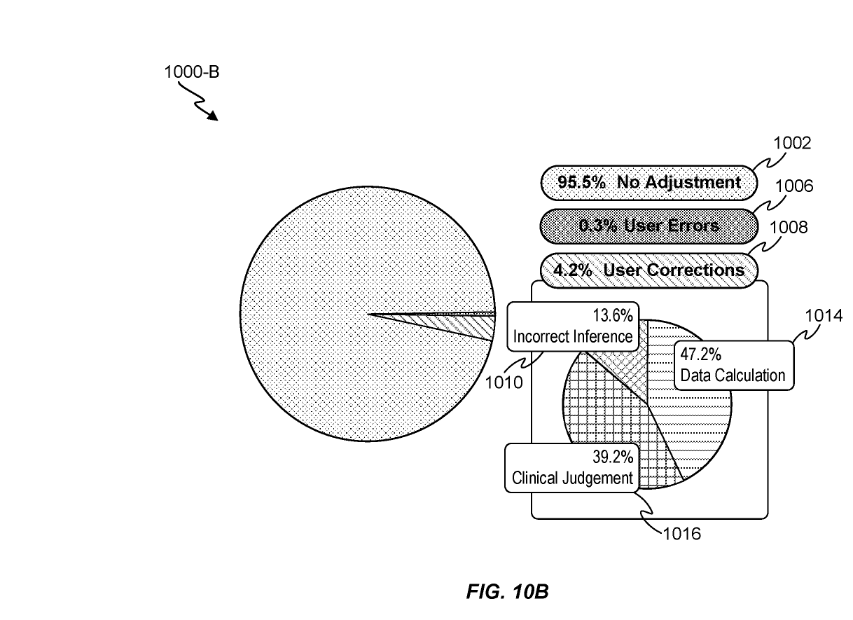

FIG. 10A illustrates a first set of metrics 1000-A related to performance of the LLE architecture in determining decision factors, in accordance with some aspects of the present disclosure. The performance of the LLE architecture 108 may be evaluated through a retrospective study conducted with 100 de-identified subject cases (50 breast cancer and 50 colon cancer). Each subject’s records were categorized into diagnosis records (up to and including the date of diagnosis) and treatment records (up to but not including the initiation of treatment). The metrics 1000-A may represent the processing of the subject’s records to generate the predicted intermediate results, which correspond to the decision factors. A user (or an expert) may then review the generated outputs, make any necessary adjustments, and refine the predicted intermediate results accordingly.

In two separate runs for 50 breast cancer and 50 colon cancer subjects, the LLE 108 retrieved total of 12,532 clinical decision factors (8,932 for breast and 3,600 for colon). Of these, 260 outputs (2.1%) were adjusted by the user—172 for breast cancer and 88 for colon cancer—leaving 97.9% of the outputs unchanged, as illustrated through no adjustments 1002. Based on further analysis, the user's adjustments may be categorized into three high-level categories including: a) study artifacts 1004; b) user errors 1006; and c) user corrections 1008.

The study artifacts 1004, accounting for 0.7% of the user’s adjustments, may refer to adjustments made due to specific content formatting issues in the study data, such as de-identified field placeholders, which may not be typically required in real-world clinical applications. The user errors 1006, comprising 0.1% of the user’s adjustments, may represent adjustments erroneously made by the user, where the modifications may not align with the intended clinical context or the subject’s true condition, resulting in incorrect changes to the outputs generated by the LLE architecture 108. Furthermore, the user corrections 1008, which make up 1.3% of the user’s adjustments, may reflect valid modifications made by the user to correct actual errors in the outputs generated by the LLE architecture 108.

The user corrections 1008 may further be divided into: a) incorrect inference 1010; b) knowledge base (KB) ambiguity 1012; and c) data calculation 1014. The incorrect inference 1010, which accounts for 40.4% of the user corrections 1008, may occur when the LLE generates a response that does not align with the user’s judgment. For example, the LLE architecture 108 may incorrectly determine that a patient is postmenopausal solely based on age, while the user finds insufficient supporting evidence to confirm this, leading to an “unknown” response. These discrepancies may be addressed by refining logic of the knowledge bases and enhancing the LLM's reasoning capabilities.

The KB ambiguity 1012, which makes up 23.0% of the user corrections 1008, may occur when the LLE’s response may be inaccurate due to unclear or overly broad extraction of decision factors from the knowledge bases. For instance, when the LLE incorrectly answers "yes" to a question about whether the patient’s breast cancer is stage III, when the answer should be more specific to stage III or IV. This ambiguity may be mitigated by refining the knowledge base and implementing more precise prompting along with few-shot learning techniques.

Moreover, the date calculation errors 1014, comprising 36.6% of the user corrections 1008, may arise when the LLE architecture 108 processes date-related factors incorrectly, for example, determining a patient is older than 65 based on their date of birth, while the correct age at diagnosis is 56. These types of errors may be commonly associated with the LLM's handling of temporal computations and may be mitigated by incorporating more deterministic date-handling functions and few-shot examples.

FIG. 10B illustrates a second set of metrics 1000-B related to performance of the LLE architecture in identifying gaps in the recommendations, in accordance with some aspects of the present disclosure. Across two runs for 50 breast cancer and 50 colon cancer patients, 2,971 workup items were provided by the LLE architecture 108 (1,423 for breast, 1,548 for colon). The user made modifications to 135 workup items, or 4.5% (51 for breast, 84 for colon), where 95.5% of the adjustments may be unchanged as illustrated by the no adjustments 1002.

In the post-study analysis, the user's adjustments may be categorized into two high-level categories including: a) user errors 1006; and b) user corrections 1008. The user corrections 1008 may account for 4.2% of the total user adjustments, while user errors 1006 may comprise 0.3% of the adjustments made. The user corrections 1008 may be categorized into three primary areas: a) incorrect inference 1010, which accounts for 13.6% of adjustments and relates to the system's misjudgment of relevant workups; b) data calculation 1014, representing 47.2% of corrections due to errors in date-related computations; and c) clinical judgment 1016, comprising 39.2% of adjustments, where the user's decision differed from the knowledge base but may have been guided based on their clinical expertise.

The first set of metrics 1000-A and the second set of metrics 1000-B provide a comprehensive evaluation of the LLE 108's performance across dimensions including retrieval of decision factors, the accuracy and relevance of recommended workups, and thoroughness in assessing the completeness of the recommended workups. This multi-faceted analysis may ensure that the LLE’s outputs align with clinical needs, thereby enhancing the decision-making process and ultimately supporting more precise and efficient patient care pathways.

Along with assessing the LLE’s capability to generate guideline-concordant recommendations, time spent by the user a non-specialist physician, unfamiliar with the subject cases may also be accessed at each time-point in the workflow. For colon cancer, median time spent by the user approves the retrieved decision factors assessed through metrics 1000-A reaches up to 2.3 minutes, followed by a median time of 2.1 minutes to finalize the recommended workup assessed at metrics 1000-B. For breast cancer, the median time comprises 5.2 and 2.1 minutes for approving the decision factors and the recommended workup, respectively.

It should be noted that the median times should be considered directional, given the retrospective study conditions. Even directionally, though, these times may be a considerable enhancement over the hours anecdotally shared by users (e.g., physicians and corresponding teams) that may be spent reviewing new subject cases in the context of guidelines.

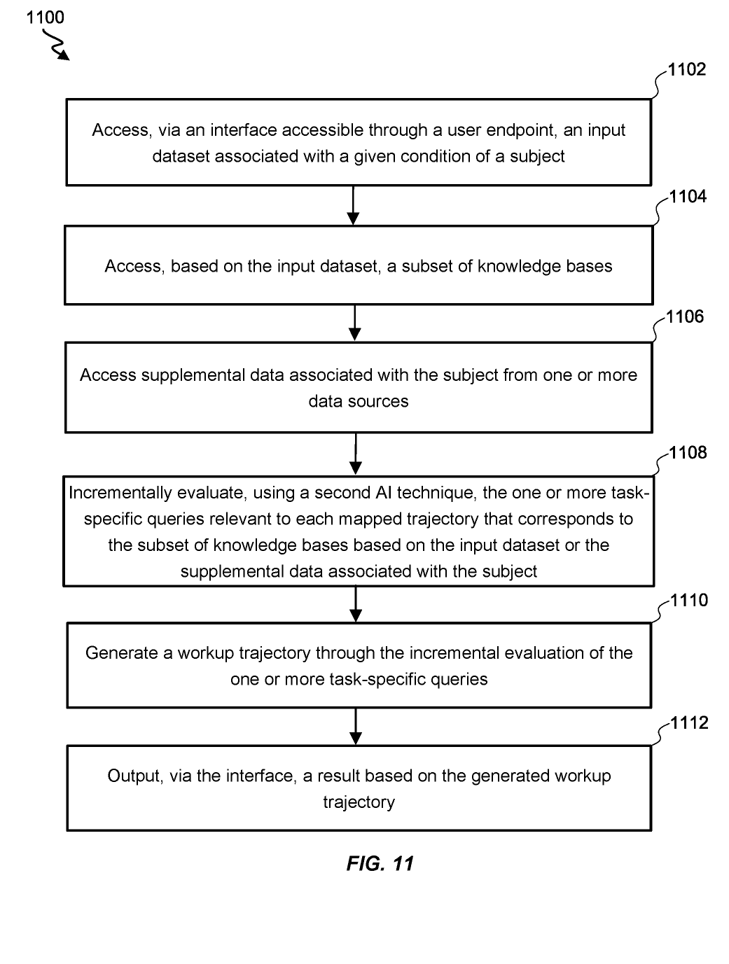

FIG. 11 illustrates an exemplary workflow 1100 of generating workup recommendations personalized in view of a given condition of a subject based on sequential and differential processing of a set of knowledge bases, in accordance with some aspects of the present disclosure. The blocks in the exemplary workflow overview 500-A and the exemplary individual workflow 500-B are illustrated in a specific order, while the order may be modified, for example, some blocks may be performed before others, and some blocks may be performed simultaneously. The block may be performed by hardware, software, or a combination thereof.

At block 1102, an input dataset may be accessed via an interface 106 accessible through a user endpoint 104. The input dataset may comprise one or more of: a diagnosis, a potential diagnosis, a medical condition, or a corresponding code associated with the given condition of the subject. In some aspects, the input dataset may further include supplemental data associated with the subject.

At block 1104, a subset of knowledge bases may be accessed from a knowledge repository 112, which may comprise a set of knowledge bases 112a-n. The subset of knowledge bases may be accessed by parsing the set of knowledge bases through application of one or more branching positions, which may be evaluated based on one or more task-specific queries. The one or more task-specific queries may be dynamically generated corresponding to the one or more variables associated with each knowledge base of the set of knowledge bases. The one or more task-specific queries may further be executed on the input dataset, filtering a plurality of knowledge bases from the set of knowledge bases relevant to the input dataset.

The one or more task-specific queries of each knowledge base from the plurality of knowledge bases may iteratively activate one or more branching positions at run time, thereby configuring a plurality of mapped trajectories aligned with the input dataset. The plurality of mapped trajectories may result in one or more actions determined in view of the given condition of the subject. The plurality of knowledge bases may then form the subset of knowledge bases. In some aspects, the plurality of knowledge bases may be hierarchically prioritized based on predefined levels of guideline authority, from which the subset of knowledge bases may then be accessed.

At block 1106, supplemental data associated with the subject may be accessed from one or more data sources, herein referred to as data sources 118. The supplemental data may include unstructured or inconsistent information specifically subject records retrieved from various sources including EHR fields, clinical text notes, scanned documents, PDFs, or other heterogeneous medical artifacts.

At block 1108, the one or more task-specific queries relevant to each knowledge base of the subset of knowledge bases may be incrementally evaluated based on the supplemental data associated with the subject. The incremental evaluation may be performed by leveraging a second AI technique implemented using the LLM 116. In some aspects, the incremental evaluation may also include executing the one or more task-specific queries on the input dataset. In some aspects, based on the evaluation of each task-specific query, a predicted intermediate result may be generated by the LLM 116. Each predicted intermediate result may include: (a) a response such as “yes,” “no,” or “unknown,” or a string or integer bundle response to the corresponding task-specific query; (b) a justification describing the basis for the response; and (c) one or more citations referencing the input dataset or the supplemental data associated with the subject.

At block 1110, a workup trajectory may be generated based on the incremental evaluation of the one or more task-specific queries. The workup trajectory may be configured by progressively refining the one or more actions associated with each mapped trajectory of a subset of mapped trajectories through the application of first-order logic. The first-order logic may be linked to the respective actions and may define the conditions under which each action may be recommended. The refined one or more actions may form part of the workup recommendations configured based on the given condition of the subject. At block 1112, a result comprising the workup recommendations may be output on the interface accessible through the user endpoint 104, enabling the user 102 to review the recommendations tailored to the given condition of the subject.

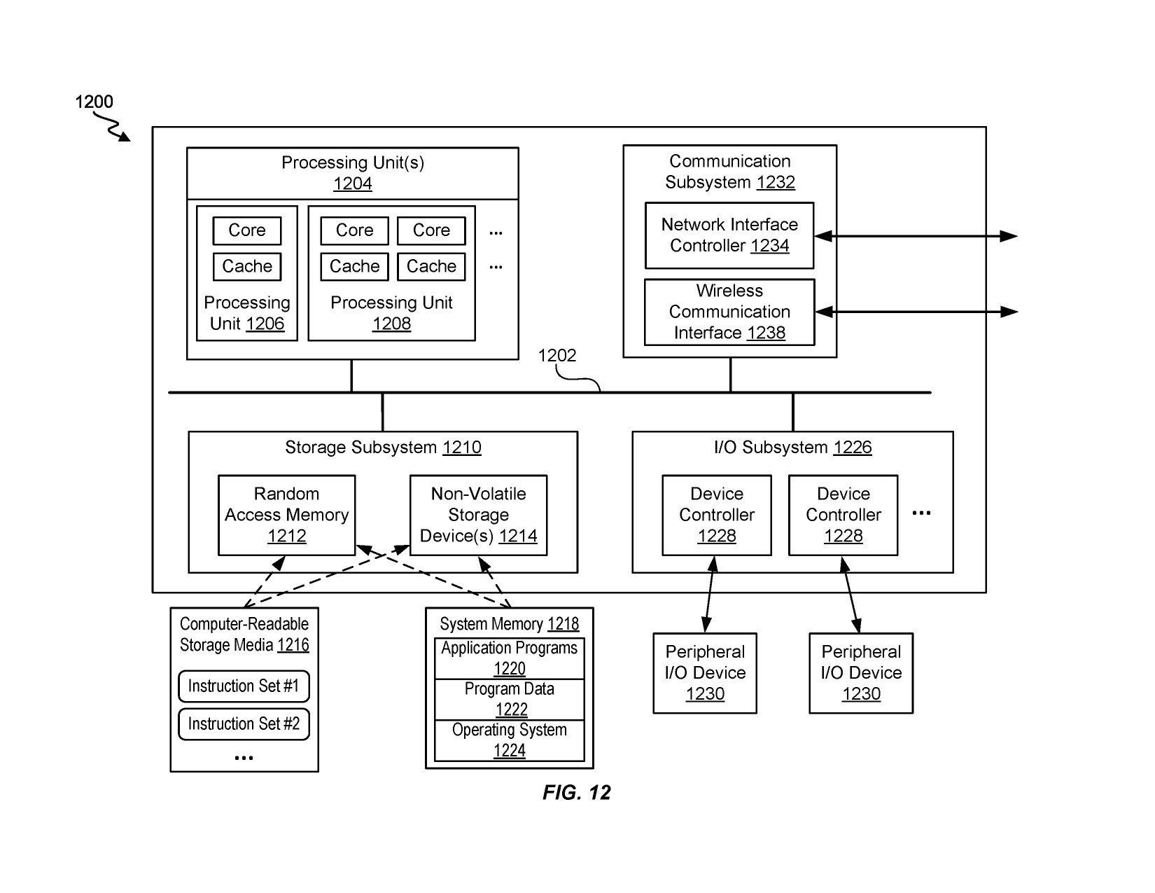

FIG. 12 illustrates a simplified diagram of an example data processing network device 1200 or system for hosting the LLE architecture 108, in accordance with some aspects of the present disclosure. The data processing network device 1200 (herein after referred to as device 1200) may correspond to any of the devices or systems within the LLE architecture 108 described above, or any other computing devices mentioned herein. Specifically, the data processing network device may include, for example, the user endpoint 104, the expert endpoint 208, and/or any processors that execute one or more workflows as disclosed herein. It will be appreciated that each of the devices referred to, that may correspond to an instance of device 1200, may be independent and unique from all other instances of device 1200 and may include fewer or additional components as those illustrated in FIG. 12.

In the example illustrated in FIG. 12, device 1200 includes processing units 1204 that communicate with several peripheral subsystems via a bus subsystem 1202. The peripheral subsystems include, for example, a storage subsystem 1210, an I/O subsystem 1226, and a communications subsystem 1232.

Bus subsystem 1202 provides a mechanism for letting the various components and subsystems of device 1200 communicate with each other. Although bus subsystem 1202 is shown schematically as a single bus, alternative embodiments of the bus subsystem may utilize multiple buses. Bus subsystem 1202 may be any of several types of bus structures including a memory bus or memory controller, a peripheral bus, and a local bus using any variety of bus architectures. The architectures may include, for example, an industry standard architecture (ISA) bus, micro channel architecture (MCA) bus, enhanced ISA (EISA) bus, video electronics standards association (VESA) local bus, and peripheral component interconnect (PCI) bus, which may be implemented as a mezzanine bus manufactured to the IEEE P1386.1 standard.

Processing unit 1204, which may be implemented as one or more integrated circuits (e.g., a conventional microprocessor or microcontroller), controls the operation of device 1200. Processing unit 1204 may be implemented as a special purpose processor, such an application-specific integrated circuit, which may be customized for a particular use and not usable for general-purpose use. One or more processors, including single core and/or multicore processors, may be included in processing unit 1204. As shown in FIG. 12, processing unit 1204 may be implemented as one or more independent processing units 1206 and/or 1208 with single or multicore processors and processor caches included in each processing unit. In other embodiments, processing unit 1204 may also be implemented as a quad-core processing unit or larger multicore designs (e.g., hexa-core processors, octa-core processors, ten-core processors, or greater).

Processing unit 1204 may execute a variety of software processes embodied in program code and may maintain multiple concurrently executing programs or processes. At any given time, some or all the program code to be executed may be resident in processor units 1204 and/or in storage subsystem 1210. In some embodiments, device 1200 may include one or more specialized processors, such as digital signal processors (DSPs), outboard processors, graphics processors, application-specific processors, and/or the like.

I/O subsystem 1226 may include device controllers 1228 for one or more user interface input devices and/or user interface output devices of the peripheral I/O device 1230. User interface input and output devices 1230 may be integral with device 1200 (e.g., integrated audio/video systems, and/or touchscreen displays) or may be separate peripheral devices which are attachable/detachable from device 1200. The I/O subsystem 1226 may provide one or several outputs to a user by converting one or several electrical signals to user perceptible and/or interpretable form, and may receive one or several inputs from the user by generating one or several electrical signals based on one or several user-caused interactions with the I/O subsystem such as the depressing of a key or button, the moving of a mouse, the interaction with a touchscreen or trackpad, the interaction of a sound wave with a microphone, or the like.

Input devices 1230 may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. Input devices 1230 may also include three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, haptic devices, and eye gaze tracking devices. Additional input devices 1230 may include, for example, motion sensing and/or gesture recognition devices that enable users to control and interact with an input device through a natural user interface using gestures and spoken commands, eye gesture recognition devices that detect eye activity from users and transform the eye gestures as input into an input device, voice recognition sensing devices that enable users to interact with voice recognition systems through voice commands, medical imaging input devices, MIDI keyboards, digital musical instruments, and the like.

Output devices 1230 may include one or more display subsystems, indicator lights, or non-visual displays such as audio output devices, etc. Display subsystems may include, for example, cathode ray tube (CRT) displays, flat-panel devices, such as those using a liquid crystal display (LCD) or plasma display, light-emitting diode (LED) displays, projection devices, touch screens, haptic devices, and the like. In general, use of the term "output device" is intended to include all possible types of devices and mechanisms for outputting information from device 1200 to a user or other computer. For example, output devices 1230 may include, without limitation, a variety of display devices that visually convey text, graphics and audio/video information such as monitors, printers, speakers, headphones, automotive navigation systems, plotters, voice output devices, and modems.

Device 1200 may comprise one or more storage subsystems 1210, comprising hardware and software components used for storing data and program instructions, such as system memory 1218 and computer-readable storage media 1216. The system memory 1218 and/or computer-readable storage media 1216 may store program instructions that are loadable and executable on processing units 1204, as well as data generated during the execution of these programs. Program instructions may include instructions to perform one or more actions or part(s) or all of one or more methods or processes described herein. For example, program instructions may include instructions for identifying and/or aligning sparse indicators. Program instructions may include instructions for bucketing or analyzing sparse indicators. Program instructions may include instructions for analyzing or counting data bucket assignments. Program instructions may include instructions for generating, transmitting, and/or receiving communications. Program instructions may include instructions for automated processing. Program instructions may include instructions for generating automated processing and/or stage results. Program instructions may include instructions for performing a workflow iteration.

Depending on the configuration and type of device 1200, system memory 1218 may be stored in volatile memory (such as random-access memory (RAM) 1212) and/or in non-volatile storage drives 1214 (such as read-only memory (ROM), flash memory, etc.) The RAM 1212 may contain data and/or program modules that are immediately accessible to and/or presently being operated and executed by processing units 1204. In some implementations, system memory 1218 may include multiple different types of memory, such as static random-access memory (SRAM) or dynamic random-access memory (DRAM). In some implementations, a basic input/output system (BIOS), containing the basic routines that help to transfer information between elements within device 1200, such as during start-up, may typically be stored in the non-volatile storage drives 1214. By way of example, and not limitation, system memory 1218 may include application programs 1220, such as user applications, Web browsers, mid-tier applications, server applications, etc., program data 1222, and an operating system 1224.

Storage subsystem 1210 also may provide one or more tangible computer-readable storage media 1216 for storing the basic programming and data constructs that provide the functionality of some embodiments. Software (programs, code modules, instructions) that when executed by a processor provide the functionality described herein may be stored in storage subsystem 1210. These software modules or instructions may be executed by processing units 1204.

Storage subsystem 1210 may also provide a repository for storing data used in accordance with the present disclosure. Storage subsystem 1210 may also include a computer-readable storage media reader that may further be connected to computer-readable storage media 1216. Together and optionally, in combination with system memory 1218, computer-readable storage media 1216 may comprehensively represent remote, local, fixed, and/or removable storage devices plus storage media for temporarily and/or more permanently containing, storing, transmitting, and retrieving computer-readable information.

Computer-readable storage media 1216 containing program code, or portions of program code, may include any appropriate media known or used in the art, including storage media and communication media, such as but not limited to, volatile and non-volatile, removable and non-removable media implemented in any method or technology for storage and/or transmission of information. This may include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media. This may also include nontangible computer-readable media, such as data signals, data transmissions, or any other medium that may be used to transmit the desired information and that may be accessed by device 1200.

By way of example, computer-readable storage media 1216 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray disk, or other optical media. Computer-readable storage media 1216 may include, but is not limited to, Zip drives, flash memory cards, universal serial bus (USB) flash drives, secure digital (SD) cards, DVD disks, digital video tape, and the like. Computer-readable storage media 1216 may also include solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magneto-resistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for device 1200.

Communications subsystem 1232 may provide a communication interface from device 1200 and remote computing devices via one or more communication networks, including local area networks (LANs), wide area networks (WANs) (e.g., the Internet), and various wireless telecommunications networks. As illustrated in FIG. 12, the communications subsystem 1232 may include, for example, one or more network interface controllers (NI Cs) 1234, such as Ethernet cards, Asynchronous Transfer Mode NICs, Token Ring NICs, and the like, as well as one or more wireless communications interfaces 1238, such as wireless network interface controllers (WNICs), wireless network adapters, and the like. Additionally, the communications subsystem 1232 may include the one or more modems (telephone, satellite, cable, ISDN), synchronous or asynchronous digital subscriber line (DSL) units, Fire Wire interfaces, USB interfaces, and the like. Communications subsystem 1232 also may include radio frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 802.11 family standards, or other mobile communication technologies, or any combination thereof), global positioning system (GPS) receiver components, and/or other components.

The various physical components of the communications subsystem 1232 may be detachable components coupled to the device 1200 via a computer network, a FireWire bus, a serial bus, or the like, and/or may be physically integrated onto a motherboard or circuit board of device 1200. Communications subsystem 1232 also may be implemented in whole or in part by software.

In some embodiments, communications subsystem 1232 may also receive input communication in the form of structured and/or unstructured data feeds, event streams, event updates, and the like, on behalf of one or more users who may use or access device 1200. For example, communications subsystem 1232 may be configured to receive data feeds in real-time from other communication services, web feeds such as Rich Site Summary (RSS) feeds, and/or real-time updates from one or more third party information sources. Additionally, communications subsystem 1232 may be configured to receive data in the form of continuous data streams, which may include event streams of real-time events and/or event updates (e.g., data vector completion, results transmission, other data transmission, report transmission, etc.). Communications subsystem 1232 may output such structured and/or unstructured data feeds, event streams, event updates, and the like to the one or more data stores that may be in communication with device 1200.

Due to the ever-changing nature of computers and networks, the description of device 1200 depicted in FIG. 12 is intended only as a specific example. Many other configurations that have more or fewer components than the device depicted in the figure are possible. For example, customized hardware might also be used and/or elements might be implemented in hardware, firmware, software, or a combination. Further, connections to other computing devices, such as network input/output devices, may be employed. Based on the disclosure and teachings provided herein, it will be appreciated that there are other ways and/or methods to implement the various embodiments.

Some embodiments of the present disclosure include a system including one or more data processors. In some embodiments, the system includes a non-transitory computer readable storage medium containing instruction which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability, or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail to avoid obscuring the embodiments.

What is claimed is:

1. A computer-implemented method comprising:
accessing, via an interface accessible through a user endpoint, an input dataset associated with a given condition of a subject;
accessing, based on the input dataset, a subset of knowledge bases, wherein the subset of knowledge bases is accessed by:
parsing each knowledge base of a set of knowledge bases through application of one or more branching positions evaluated based on one or more task-specific queries that are dynamically generated for execution on the input dataset, wherein each knowledge base of the set of knowledge bases was developed to include, at a granular level, a guideline that corresponds to one or more actions in view of a particular condition by leveraging a first artificial intelligence (AI) technique; and
dynamically stacking a plurality of knowledge bases of the set of knowledge bases based on a plurality of mapped trajectories relevant to the one or more branching positions that are activated in run-time based on the one or more task-specific queries that pertain to the input dataset, wherein each mapped trajectory of the plurality of mapped trajectories results in the one or more actions configured based on the given condition of the subject;
accessing supplemental data associated with the subject from one or more data sources;
incrementally evaluating, using a second AI technique, the one or more task-specific queries relevant to each mapped trajectory of a subset of mapped trajectories that corresponds to the subset of knowledge bases based on the input dataset or the supplemental data associated with the subject;
generating a workup trajectory through the incremental evaluation of the one or more task-specific queries, wherein the workup trajectory progressively refines the one or more actions that resulted from the subset of mapped trajectories through application of first-order logics associated with the one or more actions, wherein the first-order logics are accessed along with the one or more actions from the subset of knowledge bases; and
outputting, via the interface, a result based on the generated workup trajectory.

2. The method of claim 1, wherein the dynamic stacking further comprises:
hierarchically prioritizing the plurality of knowledge bases based on predefined levels of guideline authority; and
accessing the subset of knowledge bases from the plurality of knowledge bases based on the hierarchical prioritization.

3. The method of claim 1, wherein the incremental evaluation of the one or more task-specific queries based on the supplemental data includes:
generating, for each task-specific query, a predicted intermediate result that comprises a response to each task-specific query, a description justifying the response, and one or more citations that pertain to the input dataset or the supplemental data associated with the subject.

4. The method of claim 3, further comprising:
assigning a confidence metric to the response based on accuracy, reliability, and relevance of each predicted intermediate result, wherein the confidence metric quantifies a degree of certainty associated with the predicted intermediate result that is generated by leveraging the second AI technique.

5. The method of claim 3, further comprising:
aggregating the predicted intermediate result of each task-specific query of the one or more task-specific queries;
generating a result set based on the aggregated predicted intermediate result of each task-specific query; and
outputting the result set on the interface accessible through the user endpoint.

6. The method of claim 1, wherein the result comprises refined one or more actions based on the application of the first-order logics, wherein the refined one or more actions further comprises:
generating, for each action, a summary indicating a rationale for recommending the action that pertains to the input dataset or the supplemental data associated with the subject by leveraging the second AI technique.

7. The method of claim 1, wherein the second AI technique is implemented using a large language model (LLM), and wherein the first AI technique is similar to the second AI technique.

8. The method of claim 1, wherein the one or more task-specific queries corresponds to one or more variables associated with each of the one or more actions stored within each knowledge base of the set of knowledge bases, and wherein the one or more task-specific queries for the one or more variables are generated by leveraging the first AI technique.

9. The method of claim 8, further comprising:
accessing one or more task-specific queries based on a dynamic alignment with a given condition of the subject.

10. The method of claim 1, wherein the input dataset includes at least one of: a diagnosis, a potential diagnosis, a medical condition, or a corresponding code associated with the given condition of the subject.

11. A system comprising:
one or more data processors; and
a non-transitory computer readable storage medium containing instructions which, when executed on the one or more data processors, cause the one or more data processors to perform operations including:
accessing, via an interface accessible through a user endpoint, an input dataset associated with a given condition of a subject;
accessing, based on the input dataset, a subset of knowledge bases, wherein the subset of knowledge bases is accessed by:
parsing each knowledge base of a set of knowledge bases through application of one or more branching positions evaluated based on one or more task-specific queries that are dynamically generated for execution on the input dataset, wherein each knowledge base of the set of knowledge bases was developed to include, at a granular level, a guideline that corresponds to one or more actions in view of a particular condition by leveraging a first artificial intelligence (AI) technique; and
dynamically stacking a plurality of knowledge bases of the set of knowledge bases based on a plurality of mapped trajectories relevant to the one or more branching positions that are activated in run-time based on the one or more task-specific queries that pertain to the input dataset, wherein each mapped trajectory of the plurality of mapped trajectories results in the one or more actions configured based on the given condition of the subject;
accessing supplemental data associated with the subject from one or more data sources;
incrementally evaluating, using a second AI technique, the one or more task-specific queries relevant to each mapped trajectory of a subset of mapped trajectories that corresponds to the subset of knowledge bases based on the input dataset or the supplemental data associated with the subject;
generating a workup trajectory through the incremental evaluation of the one or more task-specific queries, wherein the workup trajectory progressively refines the one or more actions that resulted from the subset of mapped trajectories through application of first-order logics associated with the one or more actions, wherein the first-order logics are accessed along with the one or more actions from the subset of knowledge bases; and
outputting, via the interface, a result based on the generated workup trajectory.

12. A system of claim 11, wherein the dynamic stacking further comprises:
hierarchically prioritizing the plurality of knowledge bases based on predefined levels of guideline authority; and
accessing the subset of knowledge bases from the plurality of knowledge bases based on the hierarchical prioritization.

13. A system of claim 11, wherein the incremental evaluation of the one or more task-specific queries based on the supplemental data includes:
generating, for each task-specific query, a predicted intermediate result that comprises a response to each task-specific query, a description justifying the response, and one or more citations that pertain to the input dataset or the supplemental data associated with the subject.

14. A system of claim 13, further comprising:
aggregating the predicted intermediate result of each task-specific query of the one or more task-specific queries;
generating a result set based on the aggregated predicted intermediate result of each task-specific query; and
outputting the result set on the interface accessible through the user endpoint.

15. A system of claim 11, wherein the result comprises refined one or more actions based on the application of the first-order logics, wherein the refined one or more actions further comprises:
generating, for each action, a summary indicating a rationale for recommending the action that pertains to the input dataset or the supplemental data associated with the subject by leveraging the second AI technique.

16. A computer-program product tangibly embodied in a non-transitory machine-readable storage medium, including instructions configured to cause one or more data processors to perform operations including:
accessing, via an interface accessible through a user endpoint, an input dataset associated with a given condition of a subject;
accessing, based on the input dataset, a subset of knowledge bases, wherein the subset of knowledge bases is accessed by:
parsing each knowledge base of a set of knowledge bases through application of one or more branching positions evaluated based on one or more task-specific queries that are dynamically generated for execution on the input dataset, wherein each knowledge base of the set of knowledge bases was developed to include, at a granular level, a guideline that corresponds to one or more actions in view of a particular condition by leveraging a first artificial intelligence (AI) technique; and
dynamically stacking a plurality of knowledge bases of the set of knowledge bases based on a plurality of mapped trajectories relevant to the one or more branching positions that are activated in run-time based on the one or more task-specific queries that pertain to the input dataset, wherein each mapped trajectory of the plurality of mapped trajectories results in the one or more actions configured based on the given condition of the subject;
accessing supplemental data associated with the subject from one or more data sources;
incrementally evaluating, using a second AI technique, the one or more task-specific queries relevant to each mapped trajectory of a subset of mapped trajectories that corresponds to the subset of knowledge bases based on the input dataset or the supplemental data associated with the subject;
generating a workup trajectory through the incremental evaluation of the one or more task-specific queries, wherein the workup trajectory progressively refines the one or more actions that resulted from the subset of mapped trajectories through application of first-order logics associated with the one or more actions, wherein the first-order logics are accessed along with the one or more actions from the subset of knowledge bases; and
outputting, via the interface, a result based on the generated workup trajectory.

17. The computer-program product of claim 16, wherein the dynamic stacking further comprises:
hierarchically prioritizing the plurality of knowledge bases based on predefined levels of guideline authority; and
accessing the subset of knowledge bases from the plurality of knowledge bases based on the hierarchical prioritization.

18. The computer-program product of claim 16, wherein the incremental evaluation of the one or more task-specific queries based on the supplemental data includes:
generating, for each task-specific query, a predicted intermediate result that comprises a response to each task-specific query, a description justifying the response, and one or more citations that pertain to the input dataset or the supplemental data associated with the subject.

19. The computer-program product of claim 18, further comprising:
aggregating the predicted intermediate result of each task-specific query of the one or more task-specific queries;
generating a result set based on the aggregated predicted intermediate result of each task-specific query; and
outputting the result set on the interface accessible through the user endpoint.

20. The computer-program product of claim 16, wherein the result comprises refined one or more actions based on the application of the first-order logics, wherein the refined one or more actions further comprises:
generating, for each action, a summary indicating a rationale for recommending the action that pertains to the input dataset or the supplemental data associated with the subject by leveraging the second AI technique.
